(19) United States
(12) Patent Application Publication
(10) Pub. No.: US 2018/0371117 A1
(43) Pub. Date: Dec. 27, 2018

(54) SYNTHESIS AND ASSEMBLY OF CLICKABLE MICROGELS INTO CELL-LADEN POROUS SCAFFOLDS

(71) Applicant: The Regents of the University of Colorado, a body corporate, Denver, CO (US)

(72) Inventors: Kristi Anseth, Boulder, CO (US); Alexander Caldwell, Boulder, CO (US)

(21) Appl. No.: 15/995,680

(22) Filed: Jun. 1, 2018

Related U.S. Application Data
(60) Provisional application No. 62/520,275, filed on Jun. 15, 2017.

(51) Int. Cl. C08F 6/24; C07F 7/18; C08F 222/38; C12N 5/0775

(52) U.S. Cl. CPC C08F 6/24; C07F 7/1804; C08F 230/08; C12N 5/0663; C08F 222/385

(57) ABSTRACT

This invention is in the field of medicinal chemistry. The present invention provides cell-laden hydrogels and hydrogel assemblies thereof for use in tissue engineering. The present invention provides methods of producing various hydrogels and hydrogel assemblies and pharmaceutical compositions thereof. The present invention provides for a microgel comprising an encapsulated population of live primary human cells in a hydrogel comprising a polymeric network.

### CROSS-REFERENCE TO RELATED APPLICATIONS

The present application claims the benefit of U.S. Provisional Patent Application No. 62/520,275, filed on Jun. 15, 2017, which is incorporated herein by reference.

### STATEMENT OF GOVERNMENTAL SUPPORT

This invention was made with government support under grant number DE016523 awarded by the National Institutes of Health. The government has certain rights in the invention.

### FIELD OF THE INVENTION

This invention is in the field of medicinal chemistry. The present invention provides cell-laden hydrogels and hydrogel assemblies thereof for use in tissue engineering. The present invention provides methods of producing various hydrogels and hydrogel assemblies and pharmaceutical compositions thereof. The present invention provides for a microgel comprising an encapsulated population of live primary human cells in a hydrogel comprising a polymeric network.

### BACKGROUND OF THE INVENTION

Traditionally, approaches to restore tissue function have involved organ donation. However, despite attempts to encourage organ donations, there is a shortage of transplantable human tissues. Currently more than 74,000 patients in the United States are awaiting organ transplantation, while only 21,000 people receive transplants annually. Tissue engineering may provide a possible solution to alleviate the current shortage of organ donors. Tissue engineering is an interdisciplinary field that applies the principles of engineering and life sciences to develop biological substitutes, typically composed of biological and synthetic components that restore, maintain or improve tissue function. Tissue engineered products would provide a life-long therapy and would greatly reduce the hospitalization and health care costs associated with drug therapy, while simultaneously enhancing the patients' quality of life. Tissue engineering approaches have been used to create a number of biological substitutes such as bone, cardiac, smooth muscle, pancreatic, liver, tooth, retina, and skin tissues. Although tissue engineering has been relatively successful for tissues such as skin and cartilage, complex three-dimensional (3D) tissues having precisely-defined matrix properties and spatial organization of cells have not yet been generated.

There is a strong need in the art for shape- and size-controlled hydrogels in which cells have been encapsulated with homogeneous cell distribution at various viable cell densities, for methods of producing such hydrogels in the form of harvestable free units, and for tissue engineering applications in which such hydrogels mimic the architectural intricacies of physiological cell-cell interactions.

### SUMMARY OF THE INVENTION

This invention is described in preferred embodiments in the following description with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of the invention may be combined in any suitable manner in one or more embodiments. In the following description, numerous specific details are recited to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

This invention is in the field of medicinal chemistry. The present invention provides cell-laden hydrogels and hydrogel assemblies thereof for use in tissue engineering. The present invention provides methods of producing various hydrogels and hydrogel assemblies and pharmaceutical compositions thereof. The present invention provides for a microgel comprising an encapsulated population of live primary human cells in a hydrogel comprising a polymeric network.

In one embodiment, the invention contemplates a hydrogel network comprising assembled polymeric microgel particles with complementary clickable reactive surface groups. In one embodiment, said network is porous. In one embodiment, said hydrogel network comprises poly(ethylene glycol) microgel particles with dibenzocylcoctyne surface functionalities that have reacted with azide surface functionalities on poly(ethylene glycol) microgel particles. In one embodiment, live cells are encapsulated within the network. In one embodiment, said cells are primary cells. In one embodiment, said cells are human cells. In one embodiment, said human cells are mesenchymal stem cells (hMSCs). In one embodiment, said particles are composed of materials selected from the group consisting of poly(ethylene glycol), hyaluronic acid, gelatin, alginate, poly(vinyl alcohol), and polypeptides. In one embodiment, said groups react via click reactions. In one embodiment, said click reactions are selected from the group consisting of thiol-ene chemistry, Michael type additions, copper-click azide alkyne chemistries, strain-promoted alkyne-azide cycloadditions chemistry, and Diels Alder type reactions. In one embodiment, said poly(ethylene glycol) microgel particles with dibenzocylcoctyne surface functionalities comprise 20 kDa 8-arm poly(ethylene glycol) and said poly(ethylene glycol) microgel particles with azide surface functionalities comprise 4-arm 10 kDa PEG-N₃. In one embodiment, said network comprises particles with a size range between 10² nm and 10⁴ μm. In one embodiment, said network comprises particles further comprise an adhesion ligand comprising a clickable reactive group. In one embodiment, said poly(ethylene glycol) microgel particles further comprise an azide-labeled adhesion ligand.

In one embodiment, the invention contemplates a method, comprising: a) providing, i) a first group of microgel particles with a first surface functionality, ii) a second group of microgel particles with a second surface functionality, wherein said first and second surface functionalities are complementary clickable reactive surface groups, iii) a population of cells, and b) mixing said cells with said first and second microgel particles, and c) centrifuging said mixture to spontaneously form a hydrogel network encapsulating said population of cells. In one embodiment, said first group of microgel particles comprises poly(ethylene glycol) microgel particles with dibenzocylcoctyne surface functionalities and said second group of microgel particles comprises poly(ethylene glycol) microgel particles with azide surface functionalities. In one embodiment, said particles are composed of materials selected from the group consisting of poly(ethylene glycol), hyaluronic acid, gelatin, alginate, poly(vinyl alcohol), and polypeptides. In one embodiment, said groups react via click reactions. In one embodiment, said click reactions are selected from the group consisting of thiol-ene chemistry, Michael type additions, copper-click azide alkyne chemistries, strain-promoted alkyne-azide cycloadditions chemistry, and Diels Alder type reactions. In one embodiment, said network is porous.

In one embodiment, the invention contemplates a hydrogel network comprising assembled polymeric microgel particles with complementary clickable reactive surface groups and an encapsulated population of cells. In one embodiment, said network is porous. In one embodiment, said network comprises a scaffold. In one embodiment, said hydrogel network comprises poly(ethylene glycol) microgel particles with dibenzocylcoctyne surface functionalities (PEG-DBCO) that have reacted with azide surface functionalities on poly(ethylene glycol) microgel particles (PEG-N₃). In one embodiment, said cells are live cells. In one embodiment, said cells are primary cells. In one embodiment, said cells are human cells. In one embodiment, said human cells are mesenchymal stem cells (hMSCs). In one embodiment, said particles are composed of materials selected from the group consisting of poly(ethylene glycol), hyaluronic acid, gelatin, alginate, poly(vinyl alcohol), and polypeptides. In one embodiment, said groups react via click reactions. In one embodiment, said click reactions are selected from the group consisting of thiol-ene chemistry, Michael type additions, copper-click azide alkyne chemistries, strain-promoted alkyne-azide cycloadditions (SPAAC) chemistry, and Diels Alder type reactions (such as Norbornene: Tetrazine reactions). In one embodiment, said poly(ethylene glycol) microgel particles with dibenzocylcoctyne surface functionalities (PEG-DBCO) comprise 20 kDa 8-arm poly(ethylene glycol) (PEG) and said poly(ethylene glycol) microgel particles with azide surface functionalities (PEG-N₃) comprise 4-arm 10 kDa PEG-N₃. In some embodiments, it is desirable that cells are evenly distributed throughout a hydrogel. Even distribution can help provide more uniform tissue-like hydrogels that provide a more uniform environment for encapsulated cells. In some embodiments, cells are located on the surface of a hydrogel. In some embodiments, cells are located in the interior of a hydrogel. In some embodiments, the conditions under which cells are encapsulated within hydrogels are altered in order to maximize cell viability. In one embodiment, said poly(ethylene glycol) microgel particles with dibenzocylcoctyne surface functionalities (PEG-DBCO) comprise 20 kDa 8-arm poly(ethylene glycol) (PEG) and said poly(ethylene glycol) microgel particles with azide surface functionalities (PEG-N₃) comprise 4-arm 10 kDa PEG-N₃. In one embodiment, network comprises particles with a size range between 10² nm and 10⁴ μm. In one embodiment, network comprises particles with a size range between 10¹ μm and 10² μm. In one embodiment, said network comprises particles further comprise an adhesion ligand comprising a clickable reactive group. In one embodiment, said poly(ethylene glycol) microgel particles further comprise an azide-labeled adhesion ligand. In one embodiment, said poly(ethylene glycol) microgel particles further comprise adhesion promoting peptides. In one embodiment, said poly(ethylene glycol) microgel particles are highly water swollen. In one embodiment, said poly(ethylene glycol) microgel particles further comprise functionalized surfaces with free reactive groups. In one embodiment, said free reactive groups comprise complementary, bio-orthogonal, reactive groups (such as reactive click reaction groups). In one embodiment, said free reactive groups can be reacted in the presence of live cells or other biologics. In one embodiment, said microgel particles are degradable. In one embodiment, said microgel particles further comprise releasable molecules. In one embodiment, said poly(ethylene glycol) microgel particles further comprise adhesion promoting peptides. In one embodiment, said poly(ethylene glycol) microgel particles are highly water swollen. In one embodiment, said poly(ethylene glycol) microgel particles further comprise functionalized surfaces with free reactive groups. In one embodiment, said free reactive groups comprise complementary, bio-orthogonal, reactive groups (such as reactive click reaction groups). In one embodiment, said free reactive groups can be reacted in the presence of live cells or other biologics. In one embodiment, said microgel particles are degradable. In one embodiment, said microgel particles further comprise releasable molecules.

In one embodiment, the invention contemplates a hydrogel network said hydrogel network comprising poly(ethylene glycol) microgel particles with dibenzocylcoctyne surface functionalities (PEG-DBCO) that have reacted with azide surface functionalities on poly(ethylene glycol) microgel particles (PEG-N₃). In one embodiment, said poly(ethylene glycol) microgel particles with dibenzocylcoctyne surface functionalities (PEG-DBCO) comprise 20 kDa 8-arm poly(ethylene glycol) (PEG) and said poly(ethylene glycol) microgel particles with azide surface functionalities (PEG-N₃) comprise 4-arm 10 kDa PEG-N₃. In one embodiment, network comprises particles with a size range between 10¹ μm and 10² μm. In one embodiment, said poly(ethylene glycol) microgel particles further comprise an azide-labeled adhesion ligand. In one embodiment, said poly(ethylene glycol) microgel particles further comprise adhesion promoting peptides. In one embodiment, said poly(ethylene glycol) microgel particles are highly water swollen. In one embodiment, said poly(ethylene glycol) microgel particles further comprise functionalized surfaces with free reactive groups. In one embodiment, said free reactive groups comprise complementary, bio-orthogonal, reactive groups (such as reactive click reaction groups). In one embodiment, said free reactive groups can be reacted in the presence of live cells or other biologics. In one embodiment, said microgel particles are degradable. In one embodiment, said microgel particles further comprise releasable molecules.

In one embodiment, the invention contemplates a method, comprising: a) providing, i) a first group of microgel particles with a first surface functionalities, ii) a second group of microgel particles with a second surface functionalities, wherein said first and second surface functionalities are complementary clickable reactive surface groups, iii) a population of cells, and b) mixing said cells with said first and second microgel particles, and c) centrifuging said mixture to spontaneously form a hydrogel network encapsulating said population of cells. In one embodiment, said first group of microgel particles comprises poly(ethylene glycol) microgel particles with dibenzocylcoctyne surface functionalities (PEG-DBCO) and said second group of microgel particles comprises poly(ethylene glycol) microgel particles with azide surface functionalities (PEG-N₃). In one embodiment, said particles are composed of materials selected from the group consisting of poly(ethylene glycol), hyaluronic acid, gelatin, alginate, poly(vinyl alcohol), and polypeptides. In one embodiment, said groups react via click reactions. In one embodiment, said click reactions are selected from the group consisting of thiol-ene chemistry, Michael type additions, copper-click azide alkyne chemistries, strain-promoted alkyne-azide cycloadditions (SPAAC) chemistry, and Diels Alder type reactions (such as Norbornene: Tetrazine reactions). In one embodiment, said cells are live cells. In one embodiment, said cells are primary cells. In one embodiment, said cells are human cells. In one embodiment, said cells are mesenchymal stem cells (hMSCs). In one embodiment, said network is porous. In one embodiment, said centrifugation is 3,000 relative centrifugal field (RCF) at room temperature for at least 10 minutes. In one embodiment, said encapsulated cell population comprises at least 3×10⁶ cells/mL. In some embodiments, it is desirable that cells are evenly distributed throughout a hydrogel. Even distribution can help provide more uniform tissue-like hydrogels that provide a more uniform environment for encapsulated cells. In some embodiments, cells are located on the surface of a hydrogel. In some embodiments, cells are located in the interior of a hydrogel. In some embodiments, the conditions under which cells are encapsulated within hydrogels are altered in order to maximize cell viability.

In one embodiment, the invention contemplates a method, comprising: a) adding to a buffer solution: i) a first compound with a polymer unit with at least two first clickable reactive group; ii) a second compound with a polymer unit with at least two second clickable reactive group, wherein said first and second clickable reactive groups are complementary reactive groups; and iii) an adhesion ligand comprising a clickable reactive group; b) dispersing said buffer solution into a solvent solution, and c) polymerizing said solutions under nonstoichiometric conditions while mixing, wherein said polymerization yields microgel particles having excess first clickable reactive groups or second clickable reactive group surface functionalities. In one embodiment, said first compound comprises a 8-arm 20 kDa poly(ethylene glycol)-dibenzocylcoctyne (PEG-DBCO); said second compound a 4-arm 10 kDa PEG-N₃; and said labeled adhesion ligand comprises an azide-labeled adhesion ligand. In one embodiment, said first and second clickable reactive groups react via click reactions. In one embodiment, said click reactions are selected from the group consisting of thiol-ene chemistry, Michael type additions, copper-click azide alkyne chemistries, strain-promoted alkyne-azide cycloadditions (SPAAC) chemistry, and Diels Alder type reactions (such as Norbornene: Tetrazine reactions). In one embodiment, said mixing is sonicating. In one embodiment, sonicating induces formation of approximately 10¹ μm microgel particles in solution. In one embodiment, said mixing is vortexing. In one embodiment, said vortexing induces formation of approximately 10² μm microgel particles in solution. In one embodiment, said adhesion ligand comprises an RGD-containing peptide. In one embodiment, said adhesion ligand comprises fibronectin derived adhesive peptide GRGDS. In one embodiment, said buffer solution further comprises an azide functionalized fluorophore. In one embodiment, said method further comprises step d) transitioning said 10¹ μm hydrogel particles in solution into a buffer solution. In one embodiment, said method further comprises step e) combining equal volumes of said approximately 10¹ μm hydrogel particle buffer solutions having excess first or second clickable reactive group surface functionalities. In one embodiment, said method further comprises step f) centrifuging said combined buffer solutions to form a 10¹ μm hydrogel network. In one embodiment, said method further providing cells, and further comprising a step in between steps e) and f): adding said cells to said combined hydrogel particle buffer solutions. In one embodiment, said method further comprising step d) transitioning said approximately 10² μm microgel particles in solution into a buffer solution. In one embodiment, said method further comprising step e) combining equal volumes of said 10² μm microgel particle buffer solutions having excess first or second clickable reactive group surface functionalities. In one embodiment, said method further comprising and step f) centrifuging said combined buffer solutions to form a 10² μm hydrogel network. In one embodiment, said microgel particles further comprise adhesion promoting peptides. In one embodiment, said microgel particles are highly water swollen. In one embodiment, said microgel particles further comprise functionalized surfaces with free reactive groups. In one embodiment, said free reactive groups comprise complementary, bio-orthogonal, reactive groups (such as reactive click reaction groups). In one embodiment, said free reactive groups can be reacted in the presence of live cells or other biologics. In one embodiment, said microgel particles are degradable. In one embodiment, said microgel particles further comprise releasable molecules. In one embodiment, said method further providing cells and further comprising a step in between steps e) and f): adding said cells to said combined microgel particle buffer solutions. In one embodiment, said cells are live cells. In one embodiment, said cells are primary cells. In one embodiment, said cells are human cells. In one embodiment, said human cells are mesenchymal stem cells (hMSCs). In some embodiments, it is desirable that cells are evenly distributed throughout a hydrogel. Even distribution can help provide more uniform tissue-like hydrogels that provide a more uniform environment for encapsulated cells. In some embodiments, cells are located on the surface of a hydrogel. In some embodiments, cells are located in the interior of a hydrogel. In some embodiments, the conditions under which cells are encapsulated within hydrogels are altered in order to maximize cell viability.

In one embodiment, the invention contemplates a method, comprising: a) adding to a buffer solution: i) a first compound with a polymer unit with at least two first clickable reactive group; ii) a second compound with a polymer unit with at least two second clickable reactive group, wherein said first and second clickable reactive groups are complementary reactive groups; and iii) an adhesion ligand comprising a clickable reactive group, b) dispersing said buffer solution into a solvent solution, and c) polymerizing said solutions under nonstoichiometric conditions while mixing, wherein said polymerization yields microgel particles having excess first clickable reactive groups or second clickable reactive group surface functionalities. In one embodiment, said first compound comprises a 8-arm 20 kDa poly(ethylene glycol)-dibenzocylcoctyne (PEG-DBCO); said second compound a 4-arm 10 kDa PEG-N₃; and said labeled adhesion ligand comprises an azide-labeled adhesion ligand. In one embodiment, said mixing is sonicating. In one embodiment, said sonicating induces formation of approximately 10¹ μm microgel particles in solution. In one embodiment, said mixing is vortexing. In one embodiment, said vortexing induces formation of approximately 10² μm microgel particles in solution. In one embodiment, said adhesion ligand comprises an RGD-containing peptide. In one embodiment, said buffer solution further comprises a second clickable reactive group functionalized (such as an azide) fluorophore. In one embodiment, said method further comprising step d) transitioning said approximately 10¹ μm microgel particles in solution into a buffer solution, step e) combining equal volumes of said 10¹ μm microgel particle buffer solutions having excess first clickable reactive groups or second clickable reactive group surface functionalities (such as DBCO or N₃ surface functionalities) and step f) centrifuging said combined buffer solutions to spontaneously form a 10¹ μm hydrogel network. In one embodiment, said method further comprising step d) transitioning said approximately 10² μm microgel particles in solution into a buffer solution, step e) combining equal volumes of said 10² μm microgel particle buffer solutions having excess first clickable reactive groups or second clickable reactive group surface functionalities (such as DBCO or N₃ surface functionalities) and step f) centrifuging said combined buffer solutions to spontaneously form a 10² μm hydrogel network. In one embodiment, said microgel particles further comprise adhesion promoting peptides. In one embodiment, said microgel particles are highly water swollen. In one embodiment, said microgel particles further comprise functionalized surfaces with free reactive groups. In one embodiment, said free reactive groups comprise complementary, bio-orthogonal, reactive groups (such as reactive click reaction groups). In one embodiment, said free reactive groups can be reacted in the presence of live cells or other biologics. In one embodiment, said microgel particles are degradable. In one embodiment, said microgel particles further comprise releasable molecules.

In one embodiment, the invention contemplates a method, comprising: a) providing, i) poly(ethylene glycol) microgel particles with dibenzocylcoctyne surface functionalities (PEG-DBCO), ii) poly(ethylene glycol) microgel particles with azide surface functionalities (PEG-N₃), iii) a population of cells, and b) mixing said cells with said poly(ethylene glycol) microgel particles, and c) centrifuging said mixture to spontaneously (without external agent/cross-linker/enzyme ect. to cross-link the particles) form a hydrogel network encapsulating said population of cells. In one embodiment, said cells are live cells. In one embodiment, said cells are primary cells. In one embodiment, said cells are human cells. In one embodiment, said cells are mesenchymal stem cells (hMSCs). In one embodiment, said network is porous. In one embodiment, said centrifugation is 3,000 relative centrifugal field (RCF) at room temperature for at least 10 minutes. In one embodiment, said encapsulated cell population comprises at least 3×10⁶ cells/mL. In some embodiments, it is desirable that cells are evenly distributed throughout a hydrogel. Even distribution can help provide more uniform tissue-like hydrogels that provide a more uniform environment for encapsulated cells. In some embodiments, cells are located on the surface of a hydrogel. In some embodiments, cells are located in the interior of a hydrogel. In some embodiments, the conditions under which cells are encapsulated within hydrogels are altered in order to maximize cell viability.

In one embodiment, said poly(ethylene glycol) microgel particles further comprise adhesion promoting peptides. In one embodiment, said poly(ethylene glycol) microgel particles are highly water swollen. In one embodiment, said poly(ethylene glycol) microgel particles further comprise functionalized surfaces with free reactive groups. In one embodiment, said free reactive groups comprise complementary, bio-orthogonal, reactive groups (such as reactive click reaction groups). In one embodiment, said free reactive groups can be reacted in the presence of live cells or other biologics. In one embodiment, said microgel particles are degradable. In one embodiment, said microgel particles further comprise releasable molecules.

In one embodiment, the invention contemplates a method, comprising: a) adding a 8-arm 20 kDa poly(ethylene glycol)-dibenzocylcoctyne (PEG-DBCO), a 4-arm 10 kDa PEG-N₃, and an azide-labeled adhesion ligand to a buffer solution, b) dispersing said buffer solution into a solvent solution, and c) polymerizing said solutions under nonstoichiometric conditions while mixing, wherein said polymerization yields microgel particles having excess dibenzocylcoctyne or azide surface functionalities. In one embodiment, said mixing comprises sonicating. In one embodiment, said sonicating induces formation of approximately 10¹ μm microgel particles in solution. In one embodiment, said mixing comprises vortexing. In one embodiment, said vortexing induces formation of approximately 10² μm microgel particles in solution. In one embodiment, said adhesion ligand comprises an RGD-containing peptide. In one embodiment, said adhesion ligand comprises fibronectin derived adhesive peptide GRGDS. In one embodiment, said buffer solution further comprises an azide functionalized fluorophore. In one embodiment, the method further comprises a step d) transitioning said 10¹ μm hydrogel particles in solution into a buffer solution. In one embodiment, the method further comprises a step e) combining equal volumes of said approximately 10² μm hydrogel particle buffer solutions having excess dibenzocylcoctyne or azide surface functionalities. In one embodiment, the method further comprises a step f) centrifuging said combined buffer solutions to form a 10¹ μm hydrogel network. In one embodiment, the method further provides cells, and further comprising a step in between steps e) and f): adding said cells to said combined hydrogel particle buffer solutions. In one embodiment, the method further comprises a step d) transitioning said approximately 10² μm microgel particles in solution into a buffer solution. In one embodiment, the method further comprises a step e) combining equal volumes of said 10² μm microgel particle buffer solutions having excess dibenzocylcoctyne or azide surface functionalities. In one embodiment, the method further comprises a step f) centrifuging said combined buffer solutions to form a 10² μm hydrogel network. In one embodiment, the method further provides cells, and further comprising a step in between steps e) and f): adding said cells to said combined hydrogel particle buffer solutions. In one embodiment, said poly(ethylene glycol) microgel particles further comprise adhesion promoting peptides. In one embodiment, said poly(ethylene glycol) microgel particles are highly water swollen. In one embodiment, said poly(ethylene glycol) microgel particles further comprise functionalized surfaces with free reactive groups. In one embodiment, said free reactive groups comprise complementary, bio-orthogonal, reactive groups (such as reactive click reaction groups). In one embodiment, said free reactive groups can be reacted in the presence of live cells or other biologics. In one embodiment, said microgel particles are degradable. In one embodiment, said microgel particles further comprise releasable molecules. In some embodiments, it is desirable that cells are evenly distributed throughout a hydrogel. Even distribution can help provide more uniform tissue-like hydrogels that provide a more uniform environment for encapsulated cells. In some embodiments, cells are located on the surface of a hydrogel. In some embodiments, cells are located in the interior of a hydrogel. In some embodiments, the conditions under which cells are encapsulated within hydrogels are altered in order to maximize cell viability.

In one embodiment, the invention contemplates a method, comprising: a) adding 8-arm 20 kDa poly(ethylene glycol)-DBCO (PEG-DBCO), 4-arm 10 kDa PEG-N₃ and azide-labeled adhesion ligand to a buffer solution, b) dispersing said buffer solution into a solvent solution, and c) polymerizing said solutions under non-stoichiometric conditions while mixing, wherein said polymerization yields microgel particles having excess DBCO or N₃ surface functionalities. In one embodiment, said mixing is sonicating. In one embodiment, said sonicating induces formation of approximately 10¹ μm microgel particles in solution. In one embodiment, said mixing is vortexing. In one embodiment, said vortexing induces formation of approximately 10² microgel particles in solution. In one embodiment, said adhesion ligand comprises an RGD-containing peptide. In one embodiment, said buffer solution further comprises an azide functionalized fluorophore. In one embodiment, the method further comprises a step d) transitioning said approximately 10¹ μm microgel particles in solution into a buffer solution, step e) combining equal volumes of said 10¹ μm microgel particle buffer solutions having excess DBCO or N₃ surface functionalities and step f) centrifuging said combined buffer solutions to spontaneously (without external agent/cross-linker/enzyme ect. to cross-link the particles) form a 10¹ μm hydrogel network. In one embodiment, the method further provides cells, and further comprising a step in between steps e) and f): adding said cells to said combined microgel particle buffer solutions. In one embodiment, the method further comprises a step d) transitioning said approximately 10² μm microgel particles in solution into a buffer solution, step e) combining equal volumes of said 10² μm microgel particle buffer solutions having excess DBCO or N₃ surface functionalities and step f) centrifuging said combined buffer solutions to spontaneously (without external agent/cross-linker/enzyme ect. to cross-link the particles) form a 10² μm hydrogel network. In one embodiment, the method further provides cells and further comprising a step in between steps e) and f): adding said cells to said combined microgel particle buffer solutions. In one embodiment, said poly(ethylene glycol) microgel particles further comprise adhesion promoting peptides. In one embodiment, said poly(ethylene glycol) microgel particles are highly water swollen. In one embodiment, said poly(ethylene glycol) microgel particles further comprise functionalized surfaces with free reactive groups. In one embodiment, said free reactive groups comprise complementary, bio-orthogonal, reactive groups (such as reactive click reaction groups). In one embodiment, said free reactive groups can be reacted in the presence of live cells or other biologics. In one embodiment, said microgel particles are degradable. In one embodiment, said microgel particles further comprise releasable molecules. In some embodiments, it is desirable that cells are evenly distributed throughout a hydrogel. Even distribution can help provide more uniform tissue-like hydrogels that provide a more uniform environment for encapsulated cells. In some embodiments, cells are located on the surface of a hydrogel. In some embodiments, cells are located in the interior of a hydrogel. In some embodiments, the conditions under which cells are encapsulated within hydrogels are altered in order to maximize cell viability.

Those skilled in the art will recognize that the cells listed herein represent an exemplary, not comprehensive, list of cells that can be encapsulated within a precursor solution (and, therefore, eventually in a hydrogel) in accordance with the present invention.

In some embodiments, it is desirable that cells are evenly distributed throughout a hydrogel. Even distribution can help provide more uniform tissue-like hydrogels that provide a more uniform environment for encapsulated cells. In some embodiments, cells are located on the surface of a hydrogel. In some embodiments, cells are located in the interior of a hydrogel. In some embodiments, cells are layered within a hydrogel. In some embodiments, each cell layer within a hydrogel contains different cell types. In some embodiments, cell layers within a hydrogel alternate between two cell types.

In some embodiments, the conditions under which cells are encapsulated within hydrogels are altered in order to maximize cell viability. In some embodiments, for example, cell viability increases with lower polymer concentrations, lower photoinitiator concentration, and shorter UV exposure times. In some embodiments, cells located at the periphery of a hydrogel tend to have decreased viability relative to cells that are fully-encapsulated within the hydrogel. In some embodiments, conditions (e.g. pH, ionic strength, nutrient availability, temperature, oxygen availability, osmolarity, etc.) of the surrounding environment may need to be regulated and/or altered to maximize cell viability.

In some embodiments, cell viability can be measured by monitoring one of many indicators of cell viability. In some embodiments, indicators of cell viability include, but are not limited to, intracellular esterase activity, plasma membrane integrity, metabolic activity, gene expression, and protein expression. To give but one example, when cells are exposed to a fluorogenic esterase substrate (e.g. calcein AM), live cells fluoresce green as a result of intracellular esterase activity that hydrolyzes the esterase substrate to a green fluorescent product. To give another example, when cells are exposed to a fluorescent nucleic acid stain (e.g. ethidium homodimer-1), dead cells fluoresce red because their plasma membranes are compromised and, therefore, permeable to the high-affinity nucleic acid stain.

In general, the percent of cells in a precursor solution is a percent that allows for the formation of hydrogels in accordance with the present invention. In some embodiments, the percent of cells in a precursor solution that is suitable for forming hydrogels in accordance with the present invention ranges between about 0.1% w/w and about 80% w/w, between about 1.0% w/w and about 50% w/w, between about 1.0% w/w and about 40% w/w, between about 1.0% w/w and about 30% w/w, between about 1.0% w/w and about 20% w/w, between about 1.0% w/w and about 10% w/w, between about 5.0% w/w and about 20% w/w, or between about 5.0% w/w and about 10% w/w. In some embodiments, the percent of cells in a precursor solution that is suitable for forming hydrogels in accordance with the present invention is approximately 5% w/w. In some embodiments, the concentration of cells in a precursor solution that is suitable for forming hydrogels in accordance with the invention ranges between about 1×10⁵ cells/ml and 1×10⁸ cells/ml or between about 1×10⁶ cells/ml and 1×10⁷ cells/ml.

In some embodiments, a single hydrogel comprises a population of identical cells and/or cell types. In some embodiments, a single hydrogel comprises a population of cells and/or cell types that are not identical. In some embodiments, a single hydrogel may comprise at least two different types of cells. In some embodiments, a single hydrogel may comprise 3, 4, 5, 10, or more types of cells. To give but one example, in some embodiments, a single hydrogel may comprise only embryonic stem cells. In some embodiments, a single hydrogel may comprise both embryonic stem cells and hematopoietic stem cells. In some embodiments, the hydrogel may contain mesenchymal stem cells.

Other objects, advantages, and novel features, and further scope of applicability of the present invention will be set forth in part in the detailed description to follow, taken in conjunction with the accompanying drawings, and in part will become apparent to those skilled in the art upon examination of the following, or may be learned by practice of the invention. The objects and advantages of the invention may be realized and attained by means of the instrumentalities and combinations particularly pointed out in the appended claims.

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “biocompatible polymer” refers to a synthetic or natural material that is, for example, non-toxic to biological systems and/or congruent with biological processes. In this respect, biocompatibility of polymer materials denote minimal, negligible, or no risk of immunorejection, injury, damage and/or toxicity to living cells, tissues, organs, and/or biological systems. In illustrative embodiments, the biocompatible polymer is also biodegradable and/or susceptible to saccharide degradation. In illustrative embodiments, the biocompatible polymer is, for example, but not limited to, polyvinyl alcohol (PVA), polylactic acid (PLA), polyglycolic acid (PGA), poly(lactide-co-glycolide) (PLGA) and/or poly(L-lactide) (PLLA), and the like.

As used herein, the term “biodegradable” or “biodegradation” refers generally to the decomposition, i.e., breaking down, of materials, such as, for example, biological or natural material, organic matter, biocompatible materials, biocompatible polymers, and/or biosynthetic materials, when exposed to a biodegradative agent. In illustrative embodiments, “biodegradation” includes, but, is not limited to, biolytic degradation, proteolytic degradation, lipolytic degradation, saccharide degradation, degradation by microorganisms, such as, e.g., bacteria, fungi, viruses, and the like, and degradation by other natural or synthetic processes that are compatible with one or more biological systems or environments.

As used herein, the terms “extracellular matrix” or “ECM,” are used interchangeably, and encompass various liquid, gelatinous, semi-solid, or solid protein mixtures congruent with the complex extracellular environment found in many tissues. The extracellular matrix may be employed as a substrate for cell and tissue culture preparations or as a surface for cell adhesion to a hydrogel matrix. The “extracellular matrix” may also include basement membrane extract and/or Engelbreth-Holm-Swarm (EHS) matrix. In illustrative embodiments, Matrigel™ (BD Biosciences, Franklin Lakes, N.J.) is employed as the EMC, when necessary for particular applications.

As used herein, the terms “hydrogel” or “hydrogel matrix” are used interchangeably, and encompass polymer and non-polymer based hydrogels. “Hydrogel” is also meant to refer to all other hydrogel compositions disclosed herein, including hydrogels that contain polymers, copolymers, terpolymer, and complexed polymer hydrogels, i.e., hydrogels that contain one, two, three, four or more monomeric or multimeric constituent units. Along the same lines, the terms “tissue hydrogel” or “tissue matrix” refer to any composition formed into a porous matrix into which cells or tissue can grow in three dimensions. Hydrogels are typically continuous networks of hydrophilic polymers that absorb water.

As used herein, the term “organ” refers to a part or structure of the body, which is adapted for a special function or functions, and includes, but is not limited to, the skin, the lungs, the liver, the kidneys, and the bowel, including the stomach and intestines. In particular, it is contemplated that organs which are particularly susceptible to dysfunction and failure arising from an injury are amendable to tissue-engineered reconstruction and are encompassed by the term “organ.”

As used herein, the term “polymer” refers to a macromolecule made of repeating monomer or multimer units. Polymers of the present disclosure, include, but are not limited to, poly(hyaluronic acid), poly(sodium alginate), poly(ethylene glycol) (PEG), poly(lactic acid) polymers, poly(glycolic acid) polymers, poly(lactide-co-glycolides) (PLGA), poly(urethanes), poly(siloxanes) or silicones, poly(ethylene), poly(vinyl pyrrolidone), poly(2-hydroxy ethyl methacrylate), poly(N-vinyl pyrrolidone), poly(methyl methacrylate), poly(vinyl alcohol) (PVA), poly(acrylic acid), poly(vinyl acetate), polyacrylamide, poly(ethylene-co-vinyl acetate), poly(methacrylic acid), polylactic acid (PLA), poly(L-lactide) (PLLA), polyglycolic acids (PGA), nylons, polyamides, polyanhydrides, poly(ethylene-co-vinyl alcohol) (EVOH), polycaprolactone, polyvinylhydroxide, poly(ethylene oxide) (PEO), and polyorthoesters or a co-polymer or terpolymer formed from at least two or three members of the groups, respectively. The terms “hydrogel polymers” or “matrix polymer materials” refer to the materials used to make the hydrogels of the present disclosure. The terms refer to both monomeric units of the materials and the polymers or co-polymers made therefrom. Individual matrix units (monomers) or polymers can be biocompatible, biodegradable, saccharide biodegradable and/or non-biodegradable.

As used herein, the terms “saccharide degradation” or “saccharide biodegradation” or “saccharide biodegradable” refer to the decomposition, i.e., breaking down, of materials such as, for example, biocompatible polymers, co-polymers, terpolymers, and the like, when exposed to a saccharide solution. In illustrative embodiments, biocompatible polymers are susceptible to saccharide degradation by, for example, saccharide catalyzed displacement, competitive binding of saccharides, and/or sequestration of polymer units via saccharide interaction, and the like. In illustrative embodiments, the saccharides are monosaccharides, disaccharides, oligosaccharides, or polysaccharides, and the like, that bind polymers such as, for example, phenylboronate-containing copolymers (PCCs), with increased affinity compared to other PCC-polymer intermolecular interactions, e.g., PVA-PCC. The rate of degradation may be fast, e.g., degradation may take place in minutes, or slow, e.g., degradation may take place over hours, days, weeks or months, or the polymer may degrade in response to a particular saccharide concentration. In illustrative embodiments, the rate of degradation can be controlled by the type of saccharide and/or polymer that is used.

As used herein, the terms “scaffolding polymers” or “scaffolding materials” refer to the materials used to generate hydrogel scaffolds. The terms refer to both monomeric units of the materials and the polymers made therefrom. Individual scaffolding units (monomers) or polymers can be biocompatible, biodegradable, saccharide biodegradable, and/or non biodegradable.

As used herein, the term “tissue” or “tissues” refer to singular or multiply-layered structures, e.g., monolayers or stratified layers of cells, which typically constitute organ constituents. One or more different tissues may form an organ or organs. An organ may also be composed of only one type of tissue or cell. In illustrative embodiments, “tissue,” for example, emanates from one or more various types of cell layers. In this regard, tissues are composed of, for example, one or more cell-types, which include, but are not limited to, cells, muscle cells, epithelial cells, endothelia cells, stem cells, umbilical vessel cells, corneal cells, cardiomyocytes, aortic cells, corneal epithelial cells, aortic endothelial cells, fibroblasts, hair cells, keratinocytes, melanocytes, adipose cells, bone cells, osteoblasts, airway cells, microvascular cells, mammary cells, vascular cells, chondrocytes, and/or placental cells, and the like.

As used herein, microgel particles referred to as “10² μm microgels” or “100 μm microgels” refer to microgel particles ranging between 30 μm to 350 μm, with mean particle diameters of 120±60 μm and 130±80 μm for gels containing excess DBCO or azide groups, respectively. In some embodiments, particles of this 10² μm microgels size range are created when component hydrogel solutions are vortexed.

As used herein, microgel particles referred to as “10¹ μm microgels” or “10 μm microgels” refer to microgel particles ranging with average particle diameter sizes of 16±6 μm and 15±5 μm for DBCO and azide gels, respectively. In some embodiments, particles of this 10¹ μm microgel particle size range are created when component hydrogel solutions are sonicated.

As used herein, the term “live cell” is not to be limited to when every single cell is viable, but wherein a significant portion of cell population is viable. In some embodiments, live cell comprises greater than 50% viability. In some embodiments, live cell comprises greater than 80% viability. In some embodiments, live cell comprises greater than 90% viability. In preferred embodiments, live cell comprises greater than 95% viability.

As used herein, the term “viability” is not just if a population is alive or not, but can be distinguished from the all-or-nothing states of life and death by use of a quantifiable index between 0 and 1 (or 0% and 100%).

As used herein, the term “click chemistry” or more commonly called tagging, refers to a class of biocompatible reactions intended primarily to join substrates of choice with specific biomolecules. Click chemistry is not a single specific reaction, but describes a way of generating products that follow examples in nature, which also generates substances by joining small modular units. In general, click reactions usually join a biomolecule and a reporter molecule. Click chemistry is not limited to biological conditions: the concept of a “click” reaction has been used in pharmacological and various biomimetic applications. However, they have been made notably useful in the detection, localization and qualification of biomolecules. Examples of click reactions are selected from the group consisting of thiol-ene chemistry, Michael type additions, copper-click azide alkyne chemistries, strain-promoted alkyne-azide cycloadditions (SPAAC) chemistry, and Diels Alder type reactions (such as Norbornene: Tetrazine reactions)

As used herein, the term “complementary clickable reactive groups” refers to types of reactive groups that react in “click chemistry.” Non-limiting examples of “complementary clickable reactive groups” include i) azide group and strained alkyne group; ii) nitrone group and a cyclooctyne group; iii) using a nitrile oxide group (as a 1,3-dipole) and a norbornene group (as a dipolarophile); iv) oxanorbornadiene group and an azide group; v) trans-cyclooctene group and an s-tetrazine group; and vi) tertiary amine or isocyanopropanoate groups and an s-tetrazine group.

As used herein, the term “complementary clickable reactive surface groups” refers to having on the surface of a microgel at least one complementary clickable reactive group, as described above. Such groups may react with the complementary group to link microgels.

### Tissue Engineering Scaffolds

Hydrogel matrices can be used as conformable, malleable, or injectable conduits for in situ or in vivo administration of cells, drugs, or tissues to a subject. Hydrogels also function as scaffolds that facilitate in vitro and ex vivo cell growth, thereby allowing for cell-sheet and/or tissue formation prior to, or simultaneous with, administration to a subject. Synthetic hydrogels can be sterilized and do not have the associated risk of harboring contaminants, e.g., infectious agents. However, synthetic hydrogels typically do not mimic the extracellular matrix (ECM) and therefore may not properly direct cellular ingrowth or function. Hydrogels composed of an ECM imitate the native cellular environment. However, unless such hydrogels are made from autologous material, i.e., recognized as “self” by the immune system, immunorejection is possible.

Moreover, removal of the supporting matrix on which the cells or tissue constructs grow may be required for complete tissue reconstruction. Separation of the hydrogel matrix from tissue that has formed therewith typically disrupts ECM and cellular junctions of the tissue. Accordingly, tissue engineering is enhanced by the manufacture of biodegradable scaffolds that allow for in vitro, ex vivo, in vivo, or in situ confluent cell growth, such that cells, cell-sheets, and/or tissue constructs can be harvested in a non-invasive manner, e.g., by saccharide biodegradation and/or without the use of proteolytic enzymes. In this respect, the present disclosure advantageously provides hydrogel scaffolds, wherein cells, cell-sheets, and/or tissues are grown and harvested as contiguous cell-layers with intact cell-cell junctions and deposited ECM.

The harvested cells, cell-sheets, and/or tissues can be applied to various cell or tissue reconstruction applications, including, but not limited to, cell and tissue grafting, skin-grafting, allografting, wound healing grafts, aesthetic or functional re-modeling grafts, skin replacement, ocular reconstruction, liver tissue reconstruction, cardiac patching, bladder augmentation, ligament cell sheet patching, bone tissue repair and reconstruction, cartilage tissue repair and reconstruction, vasculature repair and reconstruction, thyroid tissue reconstruction, esophageal ulcer patching, neuronal tissue repair, pancreatic tissue repair, and tracheal reconstruction. Furthermore, in vitro cell culturing supports cell adhesion, cell viability, and cell proliferation on the hydrogel scaffolds disclosed herein, which, for example, demonstrates their general biocompatibility.

The biocompatibility of the hydrogel scaffolds emanate at least partially from the mild synthesis procedures described herein, which do not involve the use of harsh chemicals and/or gelation techniques. In addition, the hydrogel scaffolds possess monosaccharide inducible gel-sol phase transformability. As such, the hydrogel scaffolds are suitable for cell-sheet and tissue engineering, as well as cell immobilized applications relating thereto. The hydrogel scaffolds can be applied for immobilizing various types of mammalian cells, including but not limited to, hybridoma cells, kidney cells, Chinese hamster ovaries (CHO) cells, pancreatic islets, corneal epithelial cells, fibroblasts, chondrocytes, articular chondrocytes, neuroblasts, vascular endothelial cells, hepatocytes, esophageal epithelial cells, and erythrocytes. The immobilization can be achieved through various procedures, including but not limited to, adhesion, matrix entrapment, and microencapsulation. Hydrogels are typically composed of various monomeric constituents, and polymers, copolymers, or terpolymers thereof.

Polymer hydrogels are suitable matrix materials because they can be readily manufactured with a wide range of reproducible properties and structures. Depending on their composition, polymer matrices provide varying degrees of mechanical support for withstanding compressive and/or tensile forces. In this regard, maintaining the shape and integrity of the matrix can be important for certain tissue engineering applications, such as implanting newly formed tissue or a tissue-matrix complex into a subject. Typical tissue-matrix structures include various types of polymer hydrogels, which differ in their susceptibility to biodegradation.

The morphology of a hydrogel scaffold may also influence the development of tissue structure. The size, shape, and vascularization of various tissues, moreover, impart their functional characteristics within a biological system. Accordingly, it is important to properly design hydrogel scaffolds to facilitate a suitable range of mechanical and biological functions. Synthetic polymeric materials can be precisely controlled in material properties, quality, and mode of manufacture. Further, the present disclosure enables the production of synthetic polymers by various techniques, thereby providing for a consistent supply of such hydrogels in large quantities. The mechanical and physical properties of synthetic polymers can be readily adjusted through variation of molecular structures so as to fulfill their functions without the use of either fillers or additives.

A variety of polymers can be utilized to fabricate hydrogel matrices for cell-sheet and tissue production. These materials are typically employed as structural elements in the hydrogel, and include, but are not limited to, poly(hyaluronic acid), poly(sodium alginate), poly(ethylene glycol) (PEG), poly(lactic acid) polymers, poly(glycolic acid) polymers, poly(lactide-co-glycolides) (PLGA), poly(urethanes), poly(siloxanes) or silicones, poly(ethylene), poly(vinyl pyrrolidone), poly(2-hydroxy ethyl methacrylate), poly(N-vinyl pyrrolidone), poly(methyl methacrylate), poly(vinyl alcohol) (PVA), poly(acrylic acid), poly(vinyl acetate), polyacrylamide, poly(ethylene-co-vinyl acetate), poly(methacrylic acid), polylactic acid (PLA), poly(L-lactide) (PLLA), polyglycolic acids (PGA), nylons, polyamides, polyanhydrides, poly(ethylene-co-vinyl alcohol) (EVOH), polycaprolactone, polyvinylhydroxide, poly(ethylene oxide) (PEO). Some of these polymers are extensively used in biomedical applications such as drug delivery and are FDA approved for a variety of applications. A number of biocompatible polymers, such as, e.g., PVA, PGA, PLA, PLLA, PLGA, and other synthetic polymer tissue matrices are also known in the art.

### DESCRIPTION OF THE FIGURES

The accompanying figures, which are incorporated into and form a part of the specification, illustrate several embodiments of the present invention and, together with the description, serve to explain the principles of the invention. The figures are only for the purpose of illustrating a preferred embodiment of the invention and are not to be construed as limiting the invention.

FIG. 1 shows an inverse suspension polymerization of microgel building blocks. Scheme for microgel formation. 8arm 20 kDa PEG-DBCO, 4arm 10 kDa PEG-N₃, and azide labeled RGD were combined in PBS and dispersed in cyclohexane. Solutions were exposed to either continuous vortexing or sonication, while polymerizations were performed off-stoichiometry to yield microgels with excess DBCO or N₃ surface functionalities.

FIG. 2A-C shows the size characterization of vortexed and sonicated microgels. FIG. 2A shows fluorescently labeled (Alexa Fluor 594) microgels were imaged and sized using a custom Matlab script (scale bar 100 μm). FIG. 2B shows the size distribution of 10² μm gels with varying stoichiometry of reactive groups. Microgels exhibited a heterogeneous population for both excess DBCO and N₃ gels, with average diameters of 120 μm and 130 μm, respectively. FIG. 2C the size distribution of 10¹ μm gels with varied excess surface functionalities. 10¹ μm microgels had average diameters of 16 μm and 15 μm for the excess DBCO and excess N₃ cases, respectively.

FIG. 3A-C shows the Assembly of microgels into microporous scaffolds. FIG. 3A shows both the 10² μm and 10¹ μm particles, equal volumes of microgels containing excess DBCO or N₃ groups were combined and centrifuged to form a percolated network. Interactions between particles bearing surface DBCO and N₃ results in triazole formation and a covalently connected microgel network FIG. 3B shows the macroscopic scaffold formed from fluorescently labeled 10² μm particles (scale bar=10 mm). FIG. 3C shows the values for the average compressive moduli of networks formed from 10² μm and 10¹ μm particles. Compressive modulus was calculated from the slope of the stress-strain curve, yielding networks of 2.1 kPa and 3.3 kPa for the 10² μm and 10¹ μm particles, respectively.

FIG. 4A-C shows the pore analysis of microgel-assembled networks. FIG. 4A shows the three-dimensional images of scaffolds with a fluorescently labeled high molecular weight dextran solution to demonstrate the interconnectivity of pores within the scaffold. FIG. 4B shows the Matlab analysis of pores within microgel-assembled networks (10² μm particles shown, scale bar 100 μm). FIG. 4C shows the void fraction analysis of microgel-assembled networks. Void space was consistent among networks, with 29% and 12% negative space in the 10² μm and 10¹ μm networks, respectively.

FIG. 5A-C shows the analysis of pore dimensions in assembled networks. FIG. 5A shows the analysis of pore area within 10 μm and 10¹ μm scaffolds. 75% of pores in 10² μm particle gels were less than 3300 μm, while the corresponding percentage of pores in 10¹ μm gels were below 60 μm. FIG. 5B shows the analysis of the major axis of pores within assembled networks. The majority (75%) of pores within 10² μm networks had a major axis length smaller than 120 μm. Networks containing 10¹ μm particles had much smaller pores, with the same percentage of pores being less than 14 μm in length. FIG. 5C shows the aspect ratio of pores within microgel scaffolds. Pores in both networks had heterogeneous distributions of pore shapes, with networks consisting of 10² μm particles having an average aspect ratio of 2.1. Networks containing 10¹ μm microgels had a slightly smaller average aspect ratio of 1.9.

FIG. 6A-C shows Cell encapsulation within microporous assembled scaffolds. FIG. 6A shows representative images of cells encapsulated within 10² μm (left) and 10¹ μm (right) microgel networks. hMSCs are stained with calcein AM (green, live cells) and ethidium homodimer (red, dead cells) prior to imaging. Insert: Higher magnification image demonstrating cell morphology and interaction with microgels (gray). Scale bar denotes 10° μm. FIG. 6B shows cell staining for cytoskeletal morphology. Cells were stained with DAPI (nuclei, blue) and phalloidin (actin, green). Scale bars in inserts denote 10⁰ μm. Insert: Higher magnification image showing actin structure. Scale bar denotes 10⁰ μm. FIG. 6C shows quantification of cell viability and morphology. Viability (top) in both networks was similar, with approximately 95% of cells viable after 4 days in culture. However, cells in the 10¹ μm gel network were significantly more circular (bottom) than those encapsulated in 10² μM networks (**** denotes p<0.0001).

FIG. 7A-C shows In situ gelation data for SPAAC hydrogels at microgel conditions using FIG. 7A shows 11 mM excess DBCO and FIG. 7B shows 11 mM excess N₃. Each formulation was mixed for 10-15 seconds prior to the rheological measurement. Both graphs represent a single measurement for clarity, but each condition was repeated three times with similar results. Shear modulus (G′) is shown in black while loss modulus (G″) is shown in gray. FIG. 7C shows Bulk gels were also tested after 24 hours in PBS to assess the swollen compressive modulus. Observed moduli differences between the two conditions is very likely a result in the differing functionalities of the two PEG macromers (8 arm PEG-DBCO vs. 4 arm PEG-N3), which have different contributions to cross-linking density.

FIG. 8 shows Matlab code for analyzing particle size.

FIG. 9 shows Matlab code for analyzing pore size and dimensions.

FIG. 10 shows images of microgel assembled networks and unformed networks from homogenous particle solutions. FIG. 10 Top: Solutions (post-centrifugation) containing equal densities of a mixture of DBCO and N₃ particles (left), only DBCO functionalized particles (middle), and only N₃ functionalized particles for 10² μm (left group) and 10¹ μm (right group) cases. FIG. 10 Bottom: After multiple tube inversions a network was only observed in the heterogeneous particle mixture.

### DETAILED DESCRIPTION OF THE INVENTION

This invention is in the field of medicinal chemistry. The present invention provides cell-laden hydrogels and hydrogel assemblies thereof for use in tissue engineering. The present invention provides methods of producing various hydrogels and hydrogel assemblies and pharmaceutical compositions thereof. The present invention provides for a microgel comprising an encapsulated population of live primary human cells in a hydrogel comprising a polymeric network.

Hydrogels are a versatile class of polymeric networks that have been utilized for a wide variety of cell culture and tissue repair applications. These highly water-swollen polymer networks have served as a platform to support cell growth in vitro, and to improve viability and engraftment in cell transplantation therapies. Hydrogels can also be spatially confined as microscopic shapes (microgels) and used as structural components or delivery vehicles to affect cell function. Microgels have found numerous uses in cell culture, including as drug delivery vehicles, structural or bioactive components of bulk hydrogels, or cellular aggregates, and as 3D cell culture platforms.

More recently, however, microgels have been utilized as the building blocks for cell culture scaffolds. In this way, hydrogel scaffolds are produced via bottom-up fabrication, by assembling microgels into a fully percolated network. While traditional bulk networks have a porosity (mesh size) on the order of nanometers, microgel-assembled networks contain an inherent porosity on the size scale of tens to hundreds of microns, allowing for rapid cell proliferation, infiltration, and motility. While top-down approaches to creating porous scaffolds, that is pore formation by degrading part of a bulk network, have been traditionally used, bottom-up network fabrication can aid in the recreation of complex tissue architectures. Bottom-up approaches have already been used to create heterogeneous tissue mimics, and the inclusion of multiple “building blocks” could allow for numerous cues to be rapidly incorporated into a porous cell culture scaffold.

Previously, these building blocks have been assembled through physical entanglements or via external cross-linking agents. Herein a method to fabricate cell-laden microporous hydrogels by the co-assembly of reactive hydrogel particles with primary cells without the need for external cross-linking moieties is presented. Macromolecular poly(ethylene glycol) monomers end functionalized with azide and alkyne moieties were synthesized and reacted using an inverse suspension polymerization method to generate microgels with average diameters either on the order of 100 μm or 10 μm. Particles were synthesized with excess alkyne or azide functional groups, and then introduced into a cell suspension. Upon centrifugation, the particle-cell composite formed a macroscopic, but microporous, hydrogel via a spontaneous azide alkyne cycloaddition bio-click reaction (SPAAC). Introduction of a cell-adhesive ligand into the microsphere formulation allowed for cell-particle interactions, cell spreading and process extension. The ability to co-assemble functionalized particles with cells to create cell-laden scaffolds can lend itself to numerous tissue engineering applications.

While microporous scaffolds are increasingly used for regenerative medicine and tissue repair applications, the most common techniques to fabricate these scaffolds use templating or top-down fabrication approaches. Cytocompatible bottom-up assembly methods afford the opportunity to assemble microporous systems in the presence of cells and create complex polymer-cell composite systems in situ. Here, microgel building blocks with clickable surface groups are synthesized for the bottom-up fabrication of porous cell laden scaffolds. The facile nature of assembly allows for human mesenchymal stem cells to be incorporated throughout the porous scaffold. Particles are designed with mean diameters of ≈10 and 100 μm, and assembled to create varied microenvironments. The resulting pore sizes and their distribution significantly alter cell morphology and cytoskeletal formation. This microgel-based system provides numerous tunable properties that can be used to control multiple aspects of cellular growth and development, as well as providing the ability to recapitulate various biological interfaces.

### 1. Introduction

Hydrogels are a versatile class of polymeric networks that have been utilized for a wide variety of cell culture and tissue repair applications. These highly water-swollen polymer networks have served as a platform to support cell growth in vitro [1, 2], and to improve viability and engraftment in cell transplantation therapies [3, 4]. Additionally, hydrogels can be modified to mimic many important facets of the native extracellular matrix or selectively functionalized to present specific chemical [5-7] or mechanical [8-10] cues to trigger desired cellular responses. Hydrogels can also be spatially confined as microscopic shapes (microgels) and functionalized with specific moieties to affect cell function. Microgels have found numerous uses in cell culture, including as drug delivery vehicles [11], structural or bioactive components of hydrogels [12, 13], or cellular aggregates [14, 15], and as 3D cell culture platforms [16-18].

More recently, however, microgels have been utilized as the building blocks for cell culture scaffolds. In this way, hydrogel scaffolds are produced via bottom-up fabrication, by assembling microgels into a fully percolated network. While traditional bulk networks have a porosity (mesh size) on the order of nanometers, microgel-assembled networks contain an inherent porosity on the size scale of tens to hundreds of microns, allowing for rapid cell proliferation, infiltration, and motility [19, 20]. Top-down approaches to creating porous scaffolds, that is, pore formation by degrading part of a bulk network, have been traditionally used, and have been successful at creating biocompatible scaffolds with highly tunable pore characteristics [21, 22]. While top down approaches have proven useful in numerous applications, bottom-up network fabrication can aid in the recreation of complex tissue architectures. Bottom-up approaches have already been used to create heterogeneous tissue mimics [23], and the inclusion of multiple “building blocks” could allow for several cues to be rapidly incorporated into a porous cell culture scaffold. Elbert and colleagues have successfully demonstrated this concept, by creating microgel assembled scaffolds with three types of microgel components, functioning as structural supports, porogens, or drug delivery systems [24]. Similarly, microgels have been functionalized with cell adhesive peptides allowing for increased cell invasion to facilitate more rapid wound healing [19]. Finally, the Zhang group has explored sub-micron sized gels for similar purposes, demonstrating how particle size and interconnectivity within porous scaffolds can be utilized to control cell growth [25].

Thus, these scaffolds provide a wealth of opportunities to investigate cell interactions with both chemical and mechanical cues. Fabricating a scaffold with surface features on the size scale of a cell allows for the examination of how micro-structured environments (for example, trabecular bone or alveoli) affect cellular function and growth. The ability to co-assemble cells with microgel building blocks allows for facile creation of these environments in vitro, as well as providing numerous opportunities for cell transplantation and wound healing. Finally, these microgel building blocks can be functionalized with active moieties to impart specific cues to cells contained within the assembled network. Thus, a bottom-up type fabrication, which is presented herein, may be used, not only to create porous scaffolds, but to permit the assembly of heterogeneous particles and cells that can create varied complex culture environments.

In one embodiment, the present invention contemplates a method to fabricate cell-laden microporous hydrogels by the co-assembly of reactive hydrogel particles with primary cells. Macromolecular poly(ethylene glycol) monomers end functionalized with azide and alkyne moieties were synthesized and reacted using an inverse suspension polymerization method to generate microgels with average diameters either on the order of 100 μm or 10 μm. Particles were synthesized with excess alkyne or azide functional groups, and then introduced into a cell suspension. Upon centrifugation, the particle-cell composite formed a macroscopic, but microporous, hydrogel via a strain-promoted azide alkyne cycloaddition (SPAAC) with a cell density of 3×10⁶ cells mL⁻¹. Introduction of a fibronectin-derived adhesive ligand, GRGDS, into the microsphere formulation allowed for cell-particle interactions, cell spreading and process extension. The described microgel networks may have widespread applications in both in vitro cell culture and regenerative medicine.

### 2. Results

### 2.1 Microgel Synthesis

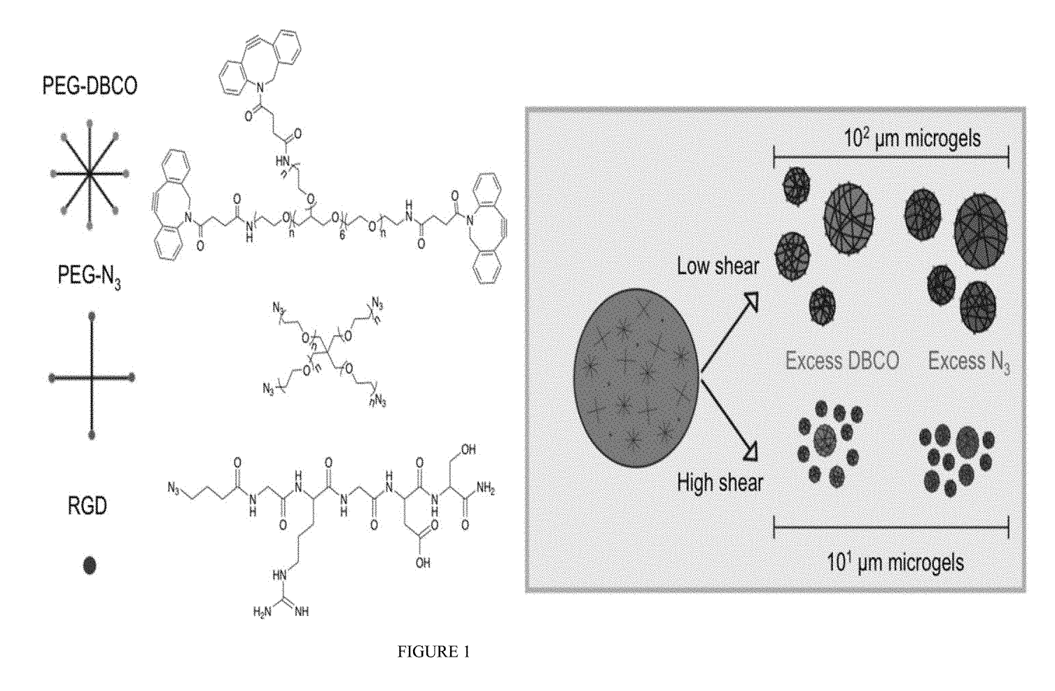

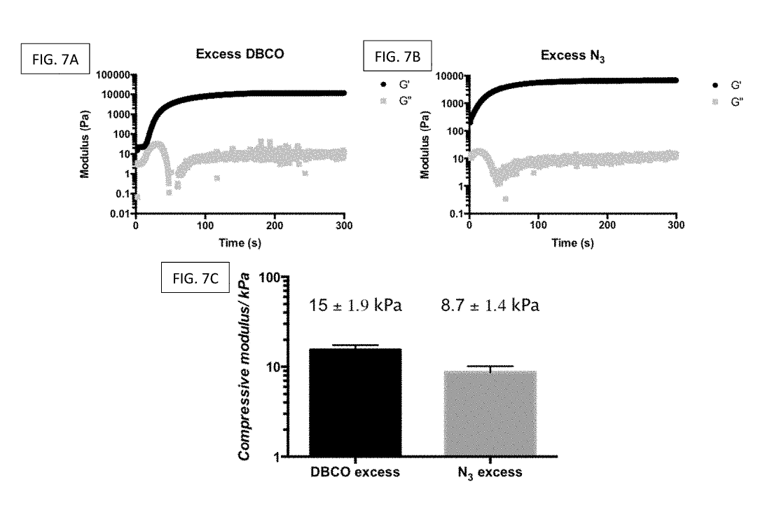

Microgels containing excess DBCO (dibenzocylcooctyne) or azide groups were formed using an inverse suspension polymerization method (FIG. 1). Briefly, aqueous precursor solutions, containing PEG-DBCO (8-arm, 20 kDa), PEG-N₃ (4-arm, 10 kDa), an azide functionalized fluorophore (AlexaFluor-594-N₃), and an azide functionalized adhesive peptide (N₃-GRGDS) were prepared with 11 mM excess of either functional group. Immediately after the addition of the limiting group (PEG-DBCO or PEG-N₃), the aqueous solution was transferred to a continuous phase of hexane, Span-80, and Tween-20. In order to control microgel size, solutions were exposed to either low shear (vortexing) or high shear (sonication) for 5 minutes, which is sufficient time for modulus evolution (FIG. 7A-C). Particles were subsequently washed with hexanes (3×), isopropanol (3×), and transitioned to PBS.

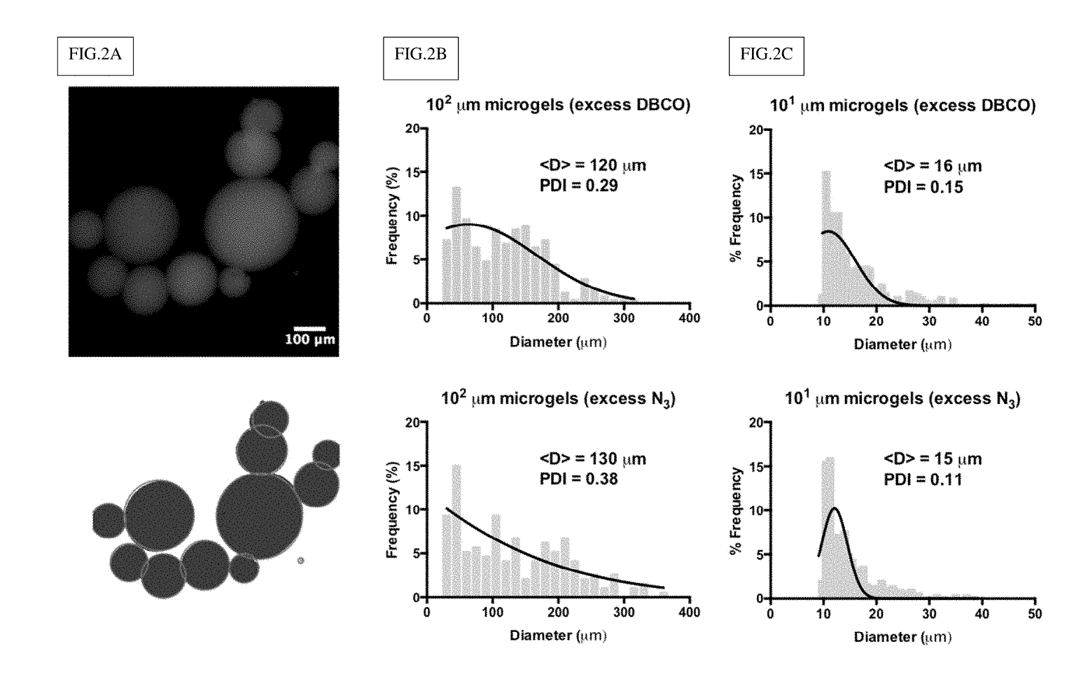

Next, the size distribution of the microgels for each formulation was categorized using a custom Matlab script (FIG. 2A-C, FIG. 8). Microgels formed using low shear (hereafter referred to as “10² μm microgels”) had a broad distribution, ranging between 30 μm to 350 μm, with mean particle diameters of 120 μm and 130 μm for gels containing excess DBCO and azide groups, respectively (FIG. 2B). Microgels formed using high shear (hereafter referred to as “10¹ μm microgels”) were an order of magnitude smaller, with average particle sizes of 16 μm and 15 μm for DBCO and azide gels, respectively (FIG. 2C). Microgel mechanical properties were approximated by measuring the moduli of bulk gels at identical cross-linking densities. Gels with excess DBCO functional groups were 15 kPa in compressive modulus, while excess azide gels had a compressive modulus of 8.7 kPa (FIG. 7C).

2.2 Assembly of Microgels into Microporous Scaffolds and Characterization

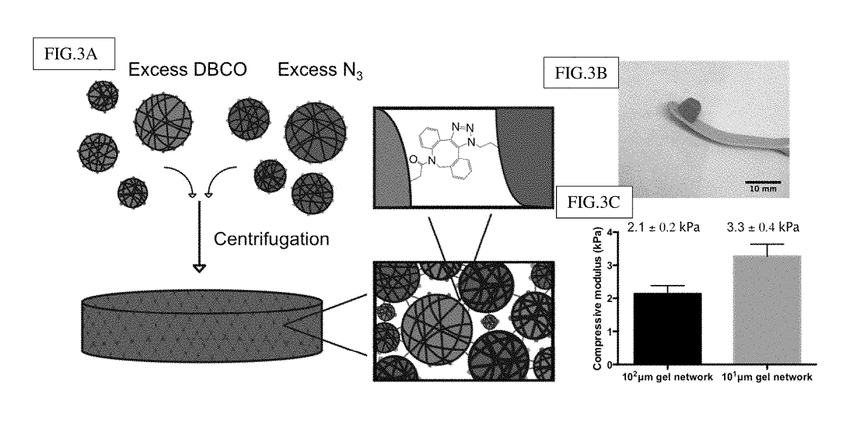

To assemble the PEG microgels into a microporous, covalently linked material, a microgel suspension was prepared containing equal densities of both DBCO and N₃ functionalized microgels. In order to increase the number of particle interactions and covalent cross-linking between microgels, solutions were concentrated via centrifuged for scaffold formation (FIG. 3A-C). While solutions containing both DBCO excess and N₃ excess microgels formed networks in both the 10² μm and 10¹ μm particle cases (FIG. 3B and FIG. 3C), solutions containing homogenous particles (only DBCO or N₃ surface reactive groups) at identical densities exposed to the same centrifugation did not form full networks (FIG. 10). The mechanical properties of the assembled gels were assessed by measuring their compressive moduli. Networks composed of 10² μm or 10¹ μm microgels formed networks with compressive moduli of approximately 2.1 kPa and 3.3 kPa, respectively (calculated via the slope of the linear stress-strain plot) (FIG. 3C). The bulk scaffold had a lower modulus (approximately 3-7 times lower) than the compressive moduli of the microgels.

Next was the categorization of the overall porosity, pore connectivity, morphology, and size distribution. Each hydrogel was first incubated in a high molecular weight fluorescein-labeled dextran solution to visualize the interconnectedness of the void space. The 250 kDa dextran readily diffused throughout the open pore structures, but did not penetrate the microgels themselves on the time scale of the experiment (FIG. 4A-C). Three-dimensional renderings of the scaffold demonstrate a continuous network of pores in both the 10² μm and 10¹ μm gel conditions. To quantify the overall porosity of the scaffolds, as well as the distribution and size of the pores, the overall void fraction and selected morphological characteristics of the pores were calculated using a custom Matlab script (FIG. 9). In brief, Z stack images were collected using a laser scanning confocal microscope of fluorescently labeled microgel assembled networks and analyzed at 12 μm intervals over 400 μm. A custom Matlab script was used to analyze the porous region in each image, identifying individual contiguous pores (FIG. 4B), and measure each pore's area, as well as major and minor axes lengths. The void space was calculated for each slice (area of pores/total area), averaged across the entire scaffold, and reported as the overall void fraction for each microgel type. The 10² μm networks had a void fraction of 29±3%, while the 10¹ μm gels had a lower void fraction of 12±2% (FIG. 4C).

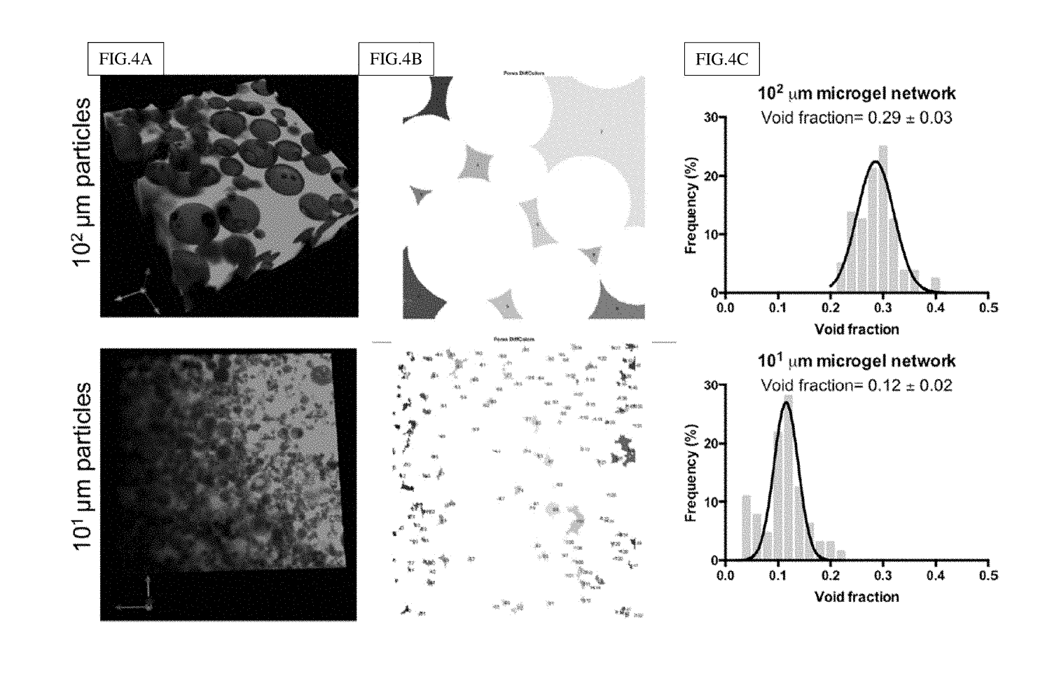

While the void space distribution was relatively non-disperse across the gels, the pore areas within the gel were relatively heterogeneous. The 10¹ μm networks contained a higher number of pores than did the 10² μm networks, while the average pore size was much larger in 10² μm networks compared to 10¹ μm networks (FIG. 5A-C). In the case of the 10² μm system, 75% of the pores were less than 3300 μm² in area, while the corresponding amount of pores were less than 60 μm² in area in the 10¹ μm system (FIG. 5A). For reference, these sizes correspond to circles with diameters of approximately 65 μm and 9 μm, respectively. However, as the pores in this gel were irregularly shaped and typically acircular, the major axes lengths were measured (FIG. 5B), and the aspect ratio (major: minor) of the pores in each gel type is reported (FIG. 5C) were assessed to better categorize the dimensions of the void space in the gels. The majority (75%) of pores in the 10² μm and 10¹ μm gels had major axes at or below 120 μm and 14.0 μm, respectively (FIG. 5B). Despite the difference in average pore size, the shape of pores (as determined by aspect ratio) was very similar in both networks (FIG. 5B). In both cases, the average aspect ratio was approximately 2, indicating elongated, acircular pore shapes (FIG. 5C).

### 2.3 Co-Assembly of Cells and Microgels

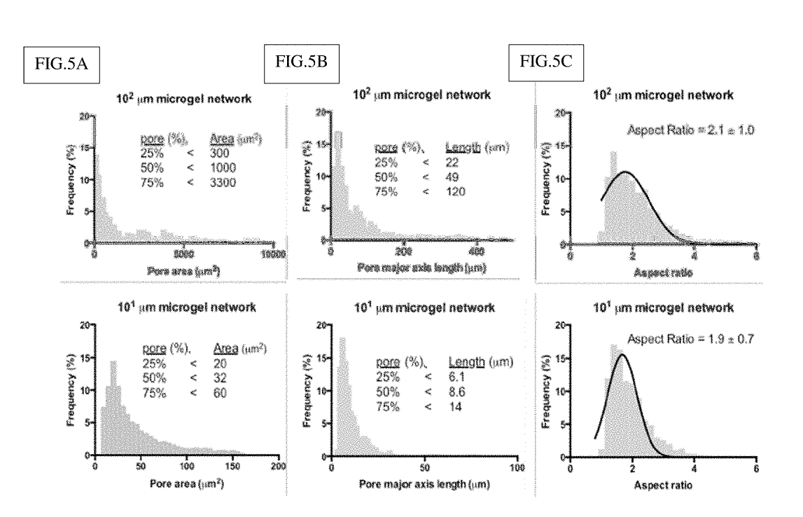

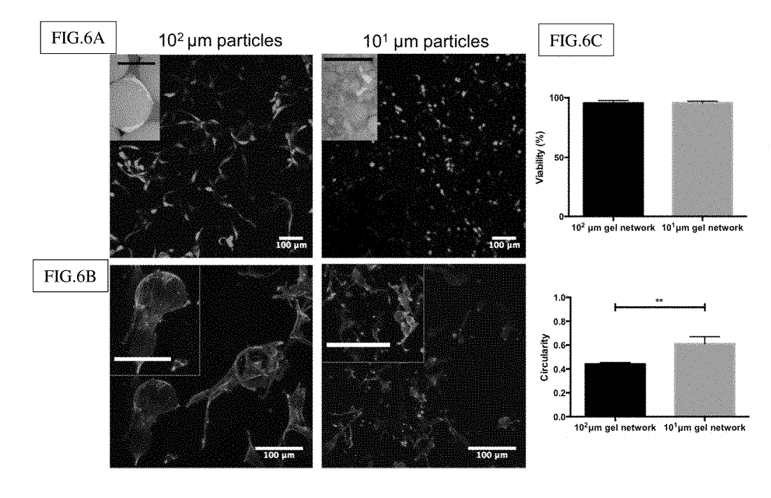

To assess the ability to co-assemble primary cells and microgels during the scaffold fabrication process, bone marrow derived mesenchymal stem cells (hMSCs) were selected as a model system. Specifically, hMSCs were suspended in the microgel formulations prior to centrifugation, and encapsulated at approximately 3×10⁶ cells mL⁻¹. Upon assembly, the cell-laden microporous scaffolds were cultured for 96 hours, and overall cell viability, cytoskeletal structure, and cell morphology were assessed (FIG. 6A-C). Cells showed high survival in both cases, with approximately 95% viability observed in both the 10² μm and 10¹ μm networks (FIG. 6C). In the 10² μm networks, hMSCs spread robustly, stretching between multiple microgels or spreading across the surface of a single microgel (FIG. 6A insert, left). Similarly, the cells in the 10¹ μm networks also adopted a spread morphology, with an increase in thin, fibular-like projections from cells (FIG. 6A insert, right). Next cellular morphology was categorized by assessing cytoskeletal structure. Cells in the 10 μm² microgel networks spread and exhibited visible actin fibers (FIG. 6B, left), while those in the 10¹ μm microgel network had only small protrusions into the matrix, with diffuse actin staining FIG. 6B, right). Finally, cell shape was quantified by analyzing area:perimeter² ratio in ImageJ, with cells in the 10¹ μm microgel networks significantly more circular than those in the 10² μm microgel networks (FIG. 6C).

### 3. Discussion

In one embodiment, the present invention contemplates a self-assembling microgel network was introduced for cell culture applications. This approach allows for the simple assembly of microgel components into a porous network without the need for porogens or post fabrication processing. The described SPAAC chemistry was used for microgel synthesis, as well as system assembly, without the addition of external initiators or cross-linking agents (FIG. 3A-C). Furthermore, stable networks did not form when only microgels containing a single surface functionality were centrifuged (FIG. 10), indicating that the networks form via covalent interactions rather than simple physical entanglements. Finally, the scaffolds demonstrated that they could be assembled in the presence of living cells at several densities, allowing for facile encapsulation and high cellular viability (FIG. 6A). By varying particle size, and thus network porosity and pore size, cell shape could be controlled, varying from a fibroblast type morphology in 10² μm microgel networks to a more rounded morphology with thin protrusions in the 10¹ μm microgel networks (FIG. 6C). This resulted in variations in cytoskeletal organization, with cells in larger pore sizes being able to form visible actin fibers, while those encapsulated in smaller pores showing only diffuse actin (FIG. 6B).

In one embodiment, the present invention contemplates modification of the microgels with the adhesive ligand RGD to allow for rapid cell spreading within the network. These cell-microgel interactions can be advantageous for network formation and engraftment in in vivo studies. Furthermore, the ability to functionalize microgels with bioactive moieties can be extended to numerous applications. In particular, functionalizing microgels with specific differentiation factors or using microgels as drug depots could allow for a new class of cell instructive materials. Microgel scaffolds have already been synthesized with deliverable growth factors [24, 26], and the fabrication of scaffolds with multiple chemical cues could allow for the creation of complex culture environments.

Beyond the inclusion of chemical cues, many material properties of this system can also be tuned. While nanoporous networks can achieve similar, or even higher void fractions [27, 28], as the current invention microgel assembled system, it has been demonstrated how particle size, and thus network pore size, affect cell growth. Both morphology and actin structure were significantly different between the 10² μm and 10¹ μm microgel networks. In the former, fibroblast morphology was observed and defined cytoskeletal actin fibers, while in the latter case cells were more contracted with thin protrusions into the network and diffuse cytosolic actin (FIG. 6A-C). This is similar to reported differences observed in cell morphology and actin structure as culture dimensionality and network elasticity is varied [29, 30]. It is possible that the smaller pore size in 10¹ μm microgel networks restricts cell spreading to thin processes, limiting cytoskeletal organization.

One advantage of this material system is the ability to manipulate the bulk versus local mechanical properties. It has been observed that a local microenvironmental stiffness greater than that of the macroscopic scaffolds, with microgels approximately 3-7 times the moduli of the bulk material. This stands to reason, as one may expect the significant percentage of void space to detract from the compressive modulus of the assembled networks. It is also possible that the dispersity of microgel sizes play an important role in determining both porosity and scaffold mechanical properties, as it is likely that this dispersity allows for tighter microgel packing than monodisperse samples. More importantly, this tunability can be highly advantageous, as one can recapitulate stiffer, yet porous, cellular environments, which may have broad reaching implications for regenerative medicine for different tissue environments (e.g., trabecular bone). Furthermore, as porous hydrogels allow for more rapid cell infiltration [31], ECM deposition [32], and a mitigated immune response [19], this system could aid in cell transplantation therapies. Thus, a microgel-assembled networks present a highly manipulatable system that could allow for a better understanding of cell function in native tissues, such as the bone marrow stem cell niche, and for in vivo tissue regeneration.

Finally, while this initial formulation relied on microgels only differing in their size and surface functionality (e.g., to allow for intraparticle cross-linking), this formulation could be readily adapted to include a more heterogeneous particle distribution to recapitulate aspects of native tissues. As material stiffness affects cellular function and fate [8, 9, 21, 33], microgels with varied stiffness, or even chemical moieties, might be included within the formulation to probe cell growth fate decisions in unique ways. While homogenous particles can be incorporated ubiquitously within the formulation, they could also be segregated to produce sub-regions within the material. Based on the nature of assembly, gradients or even distinct zones of functionalized microgels could be created via a layer-by-layer deposition method during centrifugation. These scaffolds could then be utilized to control specific cellular responses to recapitulate complex tissue interfaces both in vitro and in vivo [34]. This approach may be highly beneficial for recapitulating complex tissue interfaces (e.g., an osteochondral interface), where cell growth and fate decisions must be precisely regulated.

### 4. Conclusion

In one embodiment of the present invention, a bottom-up assembled microgel network suitable for cellular encapsulation has been designed. Clickable microgels of varied size were formed without the need for device fabrication, with varied surface functionalities allowing for spontaneous network formation. This system proved a useful cell culture platform, allowing for facile encapsulation, as well as controllable morphology, of mesenchymal stem cells. These porous scaffolds offer a high degree of tunability over both mechanical and chemical properties, and can be used to recapitulate highly varied or complex tissue environments. Collectively, the bio-click functionalized microgels and their cytocompatible assembly processes offer a unique platform to study and direct cell growth, interactions and function for both in vitro and in vivo applications.

### 5. Experimental Section

### Monomer Synthesis

Eight-arm poly(ethylene glycol) (PEG) amine (Mₙ˜20000 Da) was end functionalized with dibenzylcyclooctyne (DBCO) using standard HATU chemistry. Briefly, dibenzocyclooctyne acid (Click Chem Tools, USA), HATU (1-[Bis(dimethylamino)methylene]-1H-1,2,3-triazolo[4,5-b]pyridinium 3-oxide hexafluorophosphate) coupling agent (1:1 ratio with DBCO, Sigma Aldrich, USA), and di-isopropylethylamine (DIPEA) (2:1 ratio with DBCO, Sigma Aldrich, USA) were dissolved in N,N-dimethylformamide (peptide synthesis grade) and combined with PEG amine (1:15 ratio to DBCO). The reaction was run overnight at room temperature, subsequently concentrated under reduced pressure by rotoevaporation, and precipitated in diethyl ether at 4° C. The resulting product was resuspended in DI H₂O, dialyzed for 72 hours, and lyophilized. The extent of end-group functionalization was confirmed by ¹H NMR (Bruker AV-III) to be approximately 85%; ¹H NMR (400 MHz, CDCl₃, δ): 7.70 (d, J=7.5 Hz, 1H), 7.45 (m, 8H), 5.18 (d, J=13.8 Hz, 1H), 3.84 (q, J=4.0 Hz, 2H), 3.72 (s, 1H), 3.67 (m, PEG), 3.36 (q, J=5.3 Hz, 2H).

Four-arm poly(ethylene glycol) (PEG) azide (Mₙ˜10000 Da) was synthesized as previously reported [35]. Briefly, 4-arm poly(ethylene glycol) was dissolved in dichloromethane (DCM) and pyridine (Sigma Aldrich) and cooled to 0° C. Methanesulfonyl chloride (20 fold excess to PEG) (Sigma Aldrich) dissolved in DCM was then added dropwise, and allowed to react overnight. The product was washed with aqueous sodium bicarbonate, dried with MgSO₄, and precipitated in diethyl ether (Fisher). The mesylate activated PEG was then dissolved in anhydrous DMF along with sodium azide (5 fold excess to PEG) (Sigma Aldrich) and stirred overnight at 80° C. under argon. The product was filtered, concentrated under reduced pressure, resuspended in DI H₂O, dialyzed for 72 hours, and lyophilized. The extent of end group functionalization was confirmed by ¹H NMR (Bruker AV-III) to be >98%; ¹H NMR (400 MHz, CDCl₃, δ): 3.65 (m, PEG), 3.41 (m, 2H).

The fibronectin derived adhesive peptide GRGDS (RGD) was synthesized on a Protein Technologies Tribute Peptide Synthesizer using standard Fmoc chemistry and a Rink Amide MBHA resin (Chempep Inc, USA). Azide functionalized RGD was synthesized by coupling 4-azidobutanoic acid to the free N-terminus using standard HATU chemistry. The peptide was purified using reverse phase High Pressure Liquid Chromatography (HPLC, Waters Corporation, mobile phase: water and acetonitrile) on an XSELECT CSH C18 column and confirmed via mass spectrometry using a Voyager DE-STR MALDI-TOF (matrix-assisted laser desorption/ionization-time of flight).

### Microgel Synthesis

Off-stoichiometry monomer solutions were prepared by combining PEG-DBCO, PEG-N₃, and RGD-N₃ in Phosphate Buffered Saline (PBS) (total volume 50 μL). Solutions were made on ice with either excess DBCO or N₃ groups at a concentration of 11 mM. In the case of DBCO excess gels, 8-arm 20 kDa PEG-DBCO (3 mM), 4-arm 10 kDa PEG-N₃ (3 mM) and RGD-N₃ (1 mM) were used, while for N₃ excess gels PEG-DBCO (2 mM), PEG-N₃ (6.5 mM), and RGD-N₃ (1 mM) were used in the formulation. Each macromer solution was rapidly mixed and transferred to a continuous phase consisting of hexane with Span-80 (2.25% v/v) and Tween-20 (0.75% v/v) (20:1 ratio of continuous phase: aqueous phase). Solutions were then exposed to shear stress in the form of vortex mixing or bath sonication for 5 minutes to allow for complete polymerization (based on in situ gelation data (FIG. 7A and FIG. 7B)). Microgels were concentrated via ultracentrifugation (18,000 rcf, room temperature, 10 minutes) and washed with hexane (3×), isopropyl alcohol (3×), and PBS (1×), followed by resuspension in 1 mL of PBS. After isopropyl alcohol washes, all gels were maintained in sterile conditions.

### Microgel Size Categorization

In situ gelation data (FIG. 7A and FIG. 7B) was obtained by pipetting 20 μL of monomer solution (for either DBCO-excess or N₃-excess conditions) (cooled on ice) between the bottom plate and an 8 mm flat tool on a shear rheometer (TA DH-R3). Time sweeps were performed at 1% strain and 1 rad s⁻¹ for 300 seconds to observe full modulus evolution.

In order to assess the swollen moduli of microgels, 30 μL of monomer solutions (for either DBCO-excess or N₃-excess conditions) were pipetted into a 5 mm mold and allowed to swell overnight in PBS after formation. Swollen gels were then tested using an MTS Synergie 100. Tested gels were approximately 2 mm in height and 5.3 mm in diameter. Gels were exposed to compression up to 15% strain at a rate of 0.5 mm min⁻¹, and the compressive modulus was taken to be the slope of the reported stress-strain curve (linear region). Reported values are taken from four separate gels for each condition (DBCO-excess or N₃-excess) (FIG. 7C).

Assembly of Microgels into Macroscopic, Porous Scaffolds

Microgel-based scaffolds were assembled by co-centrifuging equal quantities of DBCO-excess and N₃-excess microgels together in 15 mL conical tubes. Particle densities of 8×10⁵ and 5×10⁷ particles/mL were used for 10² μm and 10¹ μm particle networks, respectively. The resulting microgel suspension was mixed and centrifuged (room temperature, 1,000 rcf for 10 minutes, and 3,000 rcf for 3 minutes) to form the covalently-linked microgel-assembled scaffold. The microporous gel assembly was carefully removed from the conical tube and left to equilibrate overnight in PBS, with both gels reaching final swollen volumes of 180 μL.

### Microgel Size Categorization

For imaging purposes, AlexaFluor594-N₃ (Life Technologies) was included in the DBCO-excess and N₃-excess formulations in the aqueous phase at 40 μM. After washing and transitioning to PBS, the washed microgels were diluted and placed between a glass slide and a cover slip. Gels were imaged on a Zeiss LSM710 scanning confocal microscope with a 10× objective. Images were analyzed using a custom Matlab script (FIG. 8) to quantify particle sizes and distributions, analyzing at least 200 microgels (in total) originating from three separate syntheses. PDI values are calculated by (σ*d⁻¹)², where σ is the standard deviation and d is the average particle diameter. Particle size distributions were fit to Gaussian curves using Graph Pad Prism software.

Assembly of Microgels into Macroscopic, Porous Scaffolds

Microgel-based scaffolds were assembled by co-centrifuging equal quantities of DBCO-excess and N₃-excess microgels together in 15 mL conical tubes. In both conditions, DBCO-excess and N₃-excess gels were mixed in 2 mL of PBS to reach final particle densities of 8×10⁵ and 9×10⁷ particles mL⁻¹ for 10² μm and 10¹ μm particle networks, respectively. These densities correspond to equal microgel volumes (50 μL starting monomer volume). The resulting microgel suspension was mixed and centrifuged (room temperature, 1,000 rcf for 10 minutes, and 3,000 rcf for 3 minutes) to form the covalently-linked microgel-assembled scaffold. Centrifugation speeds were chosen to ensure network formation, without limiting viability in subsequent cell studies. The microporous gel assembly was carefully removed from the conical tube and left to equilibrate overnight in PBS. Gel volumes were determined by displacement, with both 10² and 10¹ μm gel networks having average volumes of 160 μL (pre-swollen) and 180 (final swollen volume) (average measurements of at least 3 gels per condition).

### Characterization of the Microporous Gel Assembly

After equilibration in PBS, the porosity of the scaffolds containing the fluorescently labeled particles was assessed using quantitative image analysis techniques. First, images were collected on a Zeiss LSM710 scanning confocal microscope using a 10× objective; z-stacks were taken every 3-4 μm. Next, gels were incubated with high molecular weight fluorescein labeled dextran (250 kDa, Sigma Aldrich) to assess pore interconnectivity. The high molecular weight prevents dextran diffusion into the microgels, while still allowing for transport through the pores. The images were analyzed using a custom Matlab code (FIG. 9) to quantify the dimensions and size of each pore, as well as the overall scaffold porosity of the scaffolds. In brief, slices were taken every 12 μm, converted to binary, and thresholded to identify individual contiguous pores. In both cases, 400 μm stacks were imaged from three separate microgel-assembled scaffolds. Over 1500 identified pores were then categorized for each case (10² μm or 10¹ μm scaffolds). The area, major and minor axes lengths for each pore were then identified and averaged across each condition.

The macroscopic mechanical properties of the reacted microgel assemblies were tested using an MTS Synergie 100. Tested microporous hydrogels were conical, approximately 7.55 mm in height and 9 mm in diameter at the base. The microporous hydrogels were exposed to compression up to 15% strain at a rate of 0.5 mm min⁻¹, and the compressive modulus was taken to be the slope of the reported stress-strain curve (linear region). Reported compressive moduli are taken from five gels from each condition (10² μm and 10¹ μm gel networks).

### Cell Culture

Human mesenchymal stem cells (hMSCs) were isolated from bone marrow aspirates (Lonza Biosciences) as previously reported [36]. Bone marrow samples were plated on 10 mm tissue culture polystyrene plates (Corning, USA) and cultured in growth media (low-glucose DMEM (1 g L⁻¹) with 10% (v/v) fetal bovine serum, penicillin (50 U mL⁻¹), streptomycin (50 μg amphotericin B (500 ng mL⁻¹), and basic fibroblast growth factor (bFGF) (1 ng mL⁻¹)) for 72 hours at 5% CO, and 37° C. Media was aspirated to remove non-adherent cells; adherent cells were expanded in growth media, trypsinized, and frozen until use in experiments. Prior to encapsulation, cells were similarly plated, expanded (not exceeding 80% confluency), and trypsinized, with all experiments using cells at passage three.

### Assembly of Microgel-Cell Composite Scaffolds and Cell Categorization

Cell-laden microporous networks were then fabricated by co-assembling hMSCs with both DBCO-excess and N₃-excess microgels. hMSCs were mixed with DBCO-excess and N₃-excess gels and centrifuged at high speed (room temperature, 1,000 rcf for 10 minutes, and 3,000 rcf for 3 minutes) to form cell-laden gels at 3×10⁶ cells mL⁻¹. The resulting 180 μL gels were placed in wells containing hMSC experimental media (growth media without bFGF). Then cell viability and morphology was quantified to assess the potential of the network as a cell culture scaffold. Cell viability was quantified at 96 hours after encapsulation via calcein (0.5 μM, green, live) and ethidium homodimer (1 μM, red, dead) staining. Cell morphology was characterized by measuring the aspect ratio and circularity of each cell, where circularity is defined as 4n*Area*Perimeter⁻². Four separate gels for each condition (10² μm or 10¹ μm microgel networks) were analyzed with at least 100 cells from each gel (totaling 500 cells per condition). Cell circularity was averaged for each gel, and statistical analysis was then performed using an unpaired t-test with Welch's correction (to account for differing standard deviations) using Graph Pad Prism software. Finally, all images used are maximum intensity projections of 300-400 μm stacks taken with a 10× objective.

Cytoskeletal morphology of cells within microporous scaffolds was also assessed after 96 hours in culture. Microgel networks were fixed in 10% formalin (Sigma Aldrich) for 30 minutes, permeabilized in 0.1% Triton (×100, Sigma Aldrich) in PBS for 1 hour, blocked with 1% bovine serum albumin solution, and stained with DAPI (300 nM, Life Technologies) and rhodamine-phalloidin (22 nM) overnight. All images are maximum intensity projections of 200-300 μm stacks taken with a 20× objective.

Thus, specific compositions and methods of synthesis and assembly of clickable microgels into cell-laden porous scaffolds have been disclosed. It should be apparent, however, to those skilled in the art that many more modifications besides those already described are possible without departing from the inventive concepts herein. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

Although the invention has been described with reference to these preferred embodiments, other embodiments can achieve the same results. Variations and modifications of the present invention will be obvious to those skilled in the art and it is intended to cover in the appended claims all such modifications and equivalents. The entire disclosures of all applications, patents, and publications cited above, and of the corresponding application are hereby incorporated by reference.

### REFERENCES

1. Seliktar, D. (2012) “Designing Cell-Compatible Hydrogels for Biomedical Applications,” Science 336(6085), 1124-1128.
2. Guvendiren, M. and Burdick, J. A. (2013) “Engineering Synthetic Hydrogel Microenvironments to Instruct Stem Cells,” Curr. Opin. Biotechnol. 24(5), 841-846.
3. Parisi-Amon, A. et al. (2013) “Protein-Engineered Injectable Hydrogel to Improve Retention of Transplanted Adipose-Derived Stem Cells,” Adv. Healthcare Mater 2(3), 428-432.
4. Tibbitt, M. W. et al. (2015) “Progress in Material Design for Biomedical Applications,” P.N.A.S. 112(47), 14444-14451.
5. DeForest, C. A. and Tirrell, D. A. (2015) “A Photoreversible Protein-Patterning Approach for Guiding Stem Cell Fate in Three-Dimensional Gels,” Nat. Mater. 14(5), 523-531.
6. Mosiewicz, K. A. et al. (2013) “In Situ Cell Manipulation through Enzymatic Hydrogel Photopatterning,” Nat. Mater 12(11), 1072-1078.
7. Mahadik, B. P. et al. (2014) “Microfluidic Generation of Gradient Hydrogels to Modulate Hematopoietic Stem Cell Culture Environment,” Adv. Healthcare Mater. 3(3), 449-458.
8. Engler, A. J. et al. (2006) “Matrix Elasticity Directs Stem Cell Lineage Specification,” Cell 126(4), 677-689.
9. Yang, C. et al. (2014) “Mechanical Memory and Dosing Influence Stem Cell Fate,” Nat. Mater 13(6), 645-652.
10. Chaudhuri, O. et al. (2016) “Hydrogels with Tunable Stress Relaxation Regulate Stem Cell Fate and Activity,” Nat. Mater 15(3), 326-334.
11. Wang, Y. and Irvine, D. J. (2011) “Engineering Chemoattractant Gradients Using Chemokine-Releasing Polysaccharide Microspheres,” Biomaterials 32(21), 4903-4913.
12. Jha, A. K. et al. (2011) “Controlling the Adhesion and Differentiation of Mesenchymal Stem Cells Using Hyaluronic Acid-Based, Doubly Crosslinked Networks,” Biomaterials 32(10), 2466-2478.
13. Heris, H. K. et al. (2016) “Investigation of the Viability, Adhesion, and Migration of Human Fibroblasts in a Hyaluronic Acid/Gelatin Microgel-Reinforced Composite Hydrogel for Vocal Fold Tissue Regeneration,” Adv. Healthcare Mater 5(2), 255-265.
14. Nguyen, A. H. et al. (2016) “Mmp-Mediated Mesenchymal Morphogenesis of Pluripotent Stem Cell Aggregates Stimulated by Gelatin Methacrylate Microparticle Incorporation,” Biomaterials 76, 66-75.
15. Li, W. et al. (2013) “Microbead-Based Biomimetic Synthetic Neighbors Enhance Survival and Function of Rat Pancreatic 13-Cells,” Sci. Rep. 3, 2863.
16. Mao, A. S. et al. (2017) “Deterministic Encapsulation of Single Cells in Thin Tunable Microgels for Niche Modelling and Therapeutic Delivery,” Nat. Mater 16(2), 236-243.
17. Qi, H. et al. (2010) “Patterned Differentiation of Individual Embryoid Bodies in Spatially Organized 3D Hybrid Microgels,” Adv. Mater 22(46), 5276-5281.
18. Hackelbusch, S. et al. (2015) “Hybrid Microgels with Thermo-Tunable Elasticity for Controllable Cell Confinement,” Adv. Healthcare Mater 4(12), 1841-1848.
19. Griffin, D. R. et al. (2015) “Accelerated Wound Healing by Injectable Microporous Gel Scaffolds Assembled from Annealed Building Blocks,” Nat. Mater 14(7), 737-744.
20. Smith, A. W. et al. (2012) “Long-Term Culture of Hl-1 Cardiomyocytes in Modular Poly(Ethylene Glycol) Microsphere-Based Scaffolds Crosslinked in the Phase-Separated State,” Acta Biomater. 8(1), 31-40.
21. Huebsch, N. et al. (2015) “Matrix Elasticity of Void-Forming Hydrogels Controls Transplanted-Stem-Cell-Mediated Bone Formation,” Nat. Mater 14(12), 1269-1277.
22. Verbeke, C. S. and Mooney, D. J. (2015) “Injectable, Pore-Forming Hydrogels for in Vivo Enrichment of Immature Dendritic Cells,” Adv. Healthcare Mater. 4(17), 2677-2687.
23. Zamanian, B. et al. (2010) “Interface-Directed Self-Assembly of Cell-Laden Microgels,” Small 6(8), 937-944.
24. Scott, E. A. et al. (2010) “Modular Scaffolds Assembled around Living Cells Using Poly(Ethylene Glycol) Microspheres with Macroporation Via a Non-Cytotoxic Porogen,” Acta Biomater. 6(1), 29-38.
25. Shen, Z. et al. (2014) “A Thermally Responsive Cationic Nanogel-Based Platform for Three-Dimensional Cell Culture and Recovery,” RSC Advances 4(55), 29146-29156.
26. Roam, J. L. et al. (2015) “A Modular, Plasmin-Sensitive, Clickable Poly(Ethylene Glycol)-Heparin-Laminin Microsphere System for Establishing Growth Factor Gradients in Nerve Guidance Conduits,” Biomaterials 72, 112-124.
27. Kotanen, C. N. et al. (2013) “The Effect of the Physicochemical Properties of Bioactive Electroconductive Hydrogels on the Growth and Proliferation of Attachment Dependent Cells,” Biomaterials 34(27), 6318-6327.
28. Serizawa, T. et al. (2002) “Rapid and Controlled Deswelling of Porous Poly(N-Isopropylacrylamide) Hydrogels Prepared by the Templating of Interpenetrated Nanoporous Silica Particles,” J. Polym. Sci. A Polym. Chem. 40(20), 3542-3547.
29. Petrie, R. J. et al. (2012) “Nonpolarized Signaling Reveals Two Distinct Modes of 3D Cell Migration,” J. Cell Biol. 197(3), 439.
30. Rehfeldt, F. et al. (2012) “Hyaluronic Acid Matrices Show Matrix Stiffness in 2D and 3D Dictates Cytoskeletal Order and Myosin-Il Phosphorylation within Stem Cells,” Integr. Biol 4(4), 422-430.
31. Peyton, S. R. et al. (2011) “Marrow-Derived Stem Cell Motility in 3D Synthetic Scaffold Is Governed by Geometry Along with Adhesivity and Stiffness,” Biotechnol. Bioeng. 108(5), 1181-1193.
32. Hwang, Y. et al. (2010) “Interconnected Macroporous Poly(Ethylene Glycol) Cryogels as a Cell Scaffold for Cartilage Tissue Engineering,” Tissue Eng. Part A 16(10), 3033-3041.
33. Richardson, T. et al. (2016) “Capsule Stiffness Regulates the Efficiency of Pancreatic Differentiation of Human Embryonic Stem Cells,” Acta Biomater. 35, 153-165.
34. Roam, J. L. et al. (2010) “The Formation of Protein Concentration Gradients Mediated by Density Differences of Poly(Ethylene Glycol) Microspheres,” Biomaterials 31(33), 8642-8650.
35. DeForest, C. A. et al. (2009) “Sequential Click Reactions for Synthesizing and Patterning 3D Cell Microenvironments,” Nat. Mater. 8(8), 659-664.
36. Mariner, P. D. et al. (2012) “Manipulation of Mirna Activity Accelerates Osteogenic Differentiation of Hmscs in Engineered 3D Scaffolds,” J. Tissue Eng. Regen. Med. 6(4), 314-324.

We claim:

1. A hydrogel network comprising assembled polymeric microgel particles with complementary clickable reactive surface groups.

2. The hydrogel network of claim 1, wherein said network is porous.

3. The hydrogel network of claim 1, wherein said hydrogel network comprises poly(ethylene glycol) microgel particles with dibenzocylcoctyne surface functionalities that have reacted with azide surface functionalities on poly(ethylene glycol) microgel particles.

4. The hydrogel network of claim 1, wherein live cells are encapsulated within the network.

5. The hydrogel network of claim 1, wherein said cells are primary cells.

6. The hydrogel network of claim 1, wherein said cells are human cells.

7. The hydrogel network of claim 6, wherein said human cells are mesenchymal stem cells (hMSCs).

8. The hydrogel network of claim 1, wherein said particles are composed of materials selected from the group consisting of poly(ethylene glycol), hyaluronic acid, gelatin, alginate, poly(vinyl alcohol), and polypeptides.

9. The hydrogel network of claim 1, wherein said groups react via click reactions.

10. The hydrogel network of claim 9, wherein said click reactions are selected from the group consisting of thiol-ene chemistry, Michael type additions, copper-click azide alkyne chemistries, strain-promoted alkyne-azide cycloadditions chemistry, and Diels Alder type reactions.

11. The hydrogel network of claim 3, wherein said poly(ethylene glycol) microgel particles with dibenzocylcoctyne surface functionalities comprise 20 kDa 8-arm poly(ethylene glycol) and said poly(ethylene glycol) microgel particles with azide surface functionalities comprise 4-arm 10 kDa PEG-N₃.

12. The microgel of claim 1, wherein said network comprises particles with a size range between 10² nm and 10⁴ μm.

13. The microgel of claim 1, wherein said network comprises particles further comprise an adhesion ligand comprising a clickable reactive group.

14. The hydrogel network of claim 3, wherein said poly(ethylene glycol) microgel particles further comprise an azide-labeled adhesion ligand.

15. A method, comprising:
a) providing,
i) a first group of microgel particles with a first surface functionality,
ii) a second group of microgel particles with a second surface functionality, wherein said first and second surface functionalities are complementary clickable reactive surface groups,
iii) a population of cells, and
b) mixing said cells with said first and second microgel particles, and
c) centrifuging said mixture to spontaneously form a hydrogel network encapsulating said population of cells.

16. The method of claim 15, wherein said first group of microgel particles comprises poly(ethylene glycol) microgel particles with dibenzocylcoctyne surface functionalities and said second group of microgel particles comprises poly(ethylene glycol) microgel particles with azide surface functionalities.

17. The method of claim 15, wherein said particles are composed of materials selected from the group consisting of poly(ethylene glycol), hyaluronic acid, gelatin, alginate, poly(vinyl alcohol), and polypeptides.

18. The method of claim 15, wherein said groups react via click reactions.

19. The method of claim 18, wherein said click reactions are selected from the group consisting of thiol-ene chemistry, Michael type additions, copper-click azide alkyne chemistries, strain-promoted alkyne-azide cycloadditions chemistry, and Diels Alder type reactions.

20. The method of claim 15, wherein said network is porous.
